(19) United States
(12) Patent Application Publication
(10) Pub. No.: US 2005/0245508 A1
(43) Pub. Date: Nov. 3, 2005

(54) Treatment of malignant gliomas with TGF-beta inhibitors

(75) Inventors: Michael Weller, Tuebingen (DE); Sundeep Dugar, San Jose, CA (US); Linda S. Higgins, Palo Alto, CA (US); David Y. Liu, Palo Alto, CA (US); George F. Schreiner, Los Altos, CA (US); Sarvaji Chakravarty, Mountain View, CA (US)

Correspondence Address: HELLER EHRMAN LLP, 275 MIDDLEFIELD ROAD, MENLO PARK, CA, 94025-3506, US

(73) Assignee: SCIOS, Inc., Fremont, CA (US)

(21) Appl. No.: 11/021,640

(22) Filed: Dec. 22, 2004

Related U.S. Application Data
(60) Provisional application No. 60/532,346, filed on Dec. 24, 2003.

(51) Int. Cl. A61K 31/55; A61K 31/53; A61K 31/519; A61K 31/517

(52) U.S. Cl. 514/217.06; 514/241; 514/264.1; 514/266.21

(57) ABSTRACT

The invention concerns methods of treating malignant gliomas, by administering inhibitors of TGF-β the TGF-β signaling pathway, including molecules preferably binding to the type I TGF-β receptor (TGFβ-R1). Preferably, the inhibitors are non-peptide small molecules, including quinazoline derivatives. The invention also concerns methods for reversing the TGF-β-mediated effect on glioma cells to make them less refractile to signaling and other immune cells, comprising contacting a glioma cell or tissue in vivo or in vitro, with an inhibitor of TGF-β.

### BACKGROUND OF THE INVENTION

1. Field of the Invention

The present invention concerns methods of treatment of glioblastomas and other malignant gliomas associated with TGF-β signaling using transforming growth factor β (TGF-β) inhibitors. Preferably, the invention concerns methods of treating such diseases, and related conditions, by administering TGF-β inhibitors that specifically bind to the type 1 TGF-β receptor (TGFβ-R1).

2. Description of the Related Art

Transforming growth factor-beta (TGF-β) denotes a family of proteins, TGF-β1, TGF-β2, and TGF-β3, which are pleiotropic modulators of cell growth and differentiation, embryonic and bone development, extracellular matrix formation, hematopoiesis, immune and inflammatory responses (Roberts and Sporn Handbook of Experimental Pharmacology (1990) 95:419-58; Massague et al. Ann Rev Cell Biol (1990) 6:597-646). Other members of this superfamily include activin, inhibin, bone morphogenic protein, and Mullerian inhibiting substance. TGF-β initiates intracellular signaling pathways leading ultimately to the expression of genes that regulate the cell cycle, control proliferative responses, or relate to extracellular matrix proteins that mediate outside-in cell signaling, cell adhesion, migration and intercellular communication. TGF-β is known to act as a tumor suppressor at early stages of carcinogenesis, while at later stages it promotes malignant outgrowth (Cui et al., Cell (1996) 86:531-542).

TGF-β exerts its biological activities through a receptor system including the type 1 and type 2 single transmembrane TGF-β receptors (also referred to as receptor subunits) with intracellular serine-threonine kinase domains, that signal through the Smad family of transcriptional regulators. Binding of TGF-β to the extracellular domain of the type II receptor induces phosphorylation and activation of the type I receptor (TGFβ-R1) by the type B receptor (TGFβ-R2). The activated TGFβ-R1 phosphorylates a receptor-associated co-transcription transcription factor Smad2/Smad3, thereby activating it, where it binds to Smad4 in the cytoplasm. The Smad complex translocates into the nucleus, associates with a DNA-binding cofactor, such as Fast-1, binds to enhancer and suppressor regions of specific genes, and regulates transcription. The expression of these genes leads to the synthesis of cell cycle regulators that control proliferative responses or extracellular matrix proteins that mediate outside-in cell signaling, cell adhesion, migration, and intracellular communication. Other signaling pathways like the MAP kinase-ERK cascade are also activated by TGF-β signaling. For review, see, e.g. Whitman, Genes Dev. 12:2445-62 (1998); and Miyazono et al., Adv. Immunol. (2000) 75:111-57, which are expressly incorporated herein by reference. Further information about the TGF-β signaling pathway can be found, for example, in the following publications: Attisano et al., “Signal transduction by the TGF-β super-family” Science 296:1646-7 (2002); Bottinger and Bitzer, “TGF-β signaling in renal disease” Am. Soc. Nephrol. 13:2600-2610 (2002); Topper, J. N., “TGF-β in the cardiovascular system: molecular mechanisms of a context-specific growth factor” Trends Cardiovasc. Med. 10:132-7 (2000), review; Itoh et al., “Signaling of transforming growth factor-β family” Eur. J. Biochem. 267:6954-67 (2000), review.

Human glioblastoma patients experience a median survival of little more than one year with the standard treatment of surgery, radiotherapy and nitrosourea-based chemotherapy (Glioma Meta-analysis Trialists (GTM) Group. Chemotherapy in adult high-grade glioma: a systematic review and meta-analysis of individual patient data from 12 randomised trials. Lancet, 359:1011-1018 (2002)). For many years immunotherapy has been explored as an alternative approach for these tumors because human glioma patients exhibit specific deficits in their cellular immune response ex vivo (Roszman et al., Immunol. Today, 12:370-384 (1991)) and because human glioma cells are paradigmatic for the property of cancer cells to express immunosuppressive molecules. These include soluble factors such as TGF-β (Fontana et al., J. Immunol., 132:1837-1844 (1984)); prostaglandins (Fontana et al., J. Immunol., 129:2413-2419 (1982)); IL-10 (Hishii et al., Neurosurgery, 37:1160-1166 (1995)), as well as cell surface molecules such as CD70 (Wischhusen et al., Cancer Res., 62:2592-2599 (2002)) or HLA-G (Wiendl et al., J Immunol., 168:4772-4780 (2002)). The undesirable effects of TGF-β in malignant glioma are not restricted to the induction of immunosuppression in the host, but include a critical role of TGF-β in migration and invasion (Wick et al., J. Neurosci., 21:3360-3368 (2001)).

In view of the severity of glioblastoma, and the lack of satisfactory treatment options offering long term survival, there is a great need for new approaches for the treatment of this devastating disease.

### SUMMARY OF THE INVENTION

The invention concerns a novel therapeutic approach for the treatment of malignant gliomas, including glioblastomas. In particular, the invention concerns the treatment of malignant gliomas with inhibitors of members of the TGF-β signaling pathway. The invention specifically includes the treatment of malignant gliomas, including glioblastomas, with inhibitors specifically binding a TGFβ kinase receptor, such as a type 1 TGF-β receptor (TGFβ-R1).

In one aspect, the invention concerns a method for the treatment of a malignant glioma in a mammalian subject comprising administering to said subject an effective amount of a molecule that inhibits a TGFβ kinase receptor.

In a particular embodiment, the molecule used in the method of treatment is a compound of formula (1) 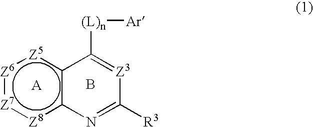
and the pharmaceutically acceptable salts and prodrug forms thereof
wherein R³ is a noninterfering substituent;
each Z is CR² or N, wherein no more than two Z positions in ring A are N, and
wherein two adjacent Z positions in ring A cannot be N;
each R² is independently a noninterfering substituent;
L is a linker;
n is 0 or 1; and
Ar′ is the residue of a cyclic aliphatic, cyclic heteroaliphatic, aromatic or heteroaromatic moiety optionally substituted with 1-3 noninterfering substituents,
or a pharmaceutically acceptable salt or prodrug form thereof.

In another embodiment, the molecule used in the treatment of the invention is a compound of formula (2) 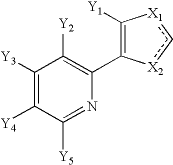
wherein
Y₁ is phenyl or naphthyl optionally substituted with one or more substituents selected from halo, alkoxy(1-6 C), alkylthio(1-6 C), alkyl(1-6 C), haloalkyl (1-6C), —O—(CH₂)ₘ-Ph, —S—(CH₂)ₘ-Ph, cyano, phenyl, and CO₂R, wherein R is hydrogen or alkyl(1-6 C), and m is 0-3; or phenyl fused with a 5- or 7-membered aromatic or non-aromatic ring wherein said ring contains up to three heteroatoms, independently selected from N, O, and S;
Y₂, Y₃, Y₄, and Y₅ independently represent hydrogen, alkyl(1-6C), alkoxy(1-6 C), haloalkyl(1-6 C), halo, NH₂, NH-alkyl(1-6C), or NH(CH₂)ₙ-Ph wherein n is 0-3; or an adjacent pair of Y₂, Y₃, Y₄, and Y₅ form a fused 6-membered aromatic ring optionally containing up to 2 nitrogen atoms, said ring being optionally substituted by one or more substituents independently selected from alkyl(1-6 C), alkoxy(a-6 C), haloalkyl(1-6 C), halo, NH₂, NH-alkyl(1-6 C), or NH(CH₂)ₙ-Ph, wherein n is 0-3, and the remainder of Y₂, Y₃, Y₄, and Y₅ represent hydrogen, alkyl(1-6 C), alkoxy(1-6C), haloalkyl(1-6 C), halo, NH₂, NH-alkyl(1-6 C), or NH(CH₂)ₙ-Ph wherein n is 0-3; and
one of X₁ and X₂ is N and the other is NR₆, wherein R₆ is hydrogen or alkyl(1-6 C), or a pharmaceutically acceptable salt or prodrug form thereof.

In a further embodiment, the molecule used in the treatment method of the invention is a compound of formula (3) 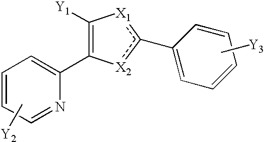
wherein
Y₁ is naphthyl, anthracenyl, or phenyl optionally substituted with one or more substituents selected from the group consisting of halo, alkoxy(1-6 C), alkylthio(1-6 C), alkyl(1-6 C), —O—(CH₂)-Ph, —S—(CH₂)ₙ-Ph, cyano, phenyl, and CO₂R, wherein R is hydrogen, or alkyl(1-6 C), and n is 0, 1, 2, or 3; or Y₁ represents phenyl fused with an aromatic or non-aromatic cyclic ring of 5-7 members wherein said cyclic ring optionally contains up to two heteroatoms, independently selected from N, O, and S;
Y₂ is H, NH(CH₂)ₙ-Ph or NH-alkyl(1-6 C), wherein n is 0, 1, 2, or 3;
Y₃ is CO₂H, CONH₂, CN, NO₂, alkylthio(1-6 C), —SO₂-alkyl(C1-6), alkoxy(C1-6), SONH₂, CONHOH, NH₂, CHO, CH₂NH₂, or CO₂R, wherein R is hydrogen or alkyl(1-6 C);
one of X₁ and X₂ is N or CR′, and other is NR′ or CHR′ wherein R′ is hydrogen, OH, alkyl(C-16), or cycloalkyl(C3-7); or when one of X₁ and X₂ is N or CR′ then the other may be S or O,
or a pharmaceutically acceptable salt or a prodrug form thereof.

In yet another embodiment, the molecule used in the treatment method of the present invention is a compound of formula (4) 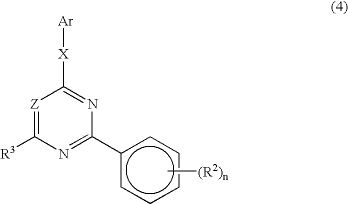
wherein
Ar represents an optionally substituted aromatic or optionally substituted heteroaromatic moiety containing 5-12 ring members wherein said heteroaromatic moiety contains one or more O, S, and/or N with a proviso that the optionally substituted Ar is not 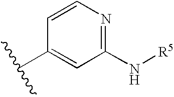
wherein R5 is H, alkyl (1-6C), alkenyl (2-6C), alkynyl (2-6C), an aromatic or heteroaromatic moiety containing 5-11 ring members;
X is NR¹, O, or S;
R¹ is H, alkyl (1-8C), alkenyl (2-8C), or alkynyl (2-8C);
Z represents N or CR⁴;
each of R³ and R⁴ is independently H, or a non-interfering substituent;
each R² is independently a non-interfering substituent; and
n is 0, 1, 2, 3, 4, or 5. In one embodiment, if n>2, and the R²'s are adjacent, they can be joined together to form a 5 to 7 membered non-aromatic, heteroaromatic, or aromatic ring containing 1 to 3 heteroatoms where each heteroatom can independently be O, N, or S,
or a pharmaceutically acceptable salt or a prodrug form thereof.

In a still further embodiment, the molecule used in the treatment method of the invention is a compound of formula (5) 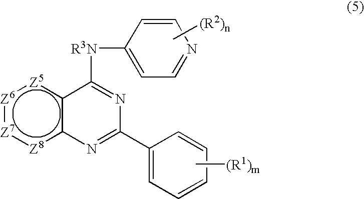
wherein
each of Z⁵, Z⁶, Z⁷ and Z⁸ is N or CH and wherein one or two Z⁵, Z⁶, Z⁷ and Z⁸ are N and wherein two adjacent Z positions cannot be N;
m and n are each independently 0-3;
R¹ is halo, alkyl, alkoxy or alkyl halide and wherein two adjacent R¹ groups may be joined to form an aliphatic heterocyclic ring of 5-6 members;
R² is a noninterfering substituent; and
R³ is H or CH₃,
or a pharmaceutically acceptable salt or a prodrug form thereof.

In another aspect, the invention concerns a method for reversing a TGF-β-mediated effect on a gene associated with a malignant glioma, comprising contacting a cell comprising such gene with a non-peptide small molecule inhibitor of TGF-β that specifically binds to a TGFβ-R1 receptor kinase present in the cell. In preferred embodiment, the small molecule inhibitor is a compound of formula (1)-(5).

### BRIEF DESCRIPTION OF THE DRAWINGS

FIG. 1. Prevention of the growth inhibitory effects of recombinant and glioma-derived TGF-b₁ and TGF-b₂ by a TGF-β inhibitor (Compound No. 79 in Table 2). A. CCL64 cells were exposed to human recombinant TGF-b₁ (filled symbols) or TGF-b₂ (open symbols) (10 ng/ml) in the absence or presence of increasing concentrations of Comopund No. 79 for 72 h. B. CCL64 cells were maintained in serum-free medium containing heat-activated glioma cell SN (1:1) in the absence or presence of Compound No. 79 for 72 h. Growth was assessed by crystal violet assay (mean and SD, n=3).

FIG. 2. Abrogation of autocrine TGF-β signaling in glioma cells by Compound No. 79. A. The cells were seeded at 10⁴ cells/well in 96 well plates and cultured in the absence or presence of Compound No. 79 for 48 h in serum-free medium. Growth was assessed by [methyl-³H]-thymidine incorporation at 48 h (*p<0.05, t-test). B. Lysates from untreated glioma cells or cells preexposed to Compound No. 79 (1 μM) for 24 h or exposed to TGF-b₂ (5 ng/ml) for 1 h or both were assessed for the levels of p-Smad2 or total Smad2/3. Note that the antibodies were specific to p-Smad2 and total Smad 2 and 3, respectively.

FIG. 3. Modulation of allogeneic anti-glioma immune responses by Compound No. 79 involves TGF-β antagonism. A. The lytic activity against LN-308 targets of PBL (squares) or purified T cells (triangles) preincubated with irradiated LN-308 cells in the absence (open symbols) or presence (filled symbols) of Compound No. 79 (1 μM) was determined in ⁵¹Cr release assays. B-D. PBL were cultured in the absence (left) or presence (right) of irradiated LN-308 cells for 5 days. The cultures contained Compound No. 79 (1 μM) (filled bars) or not (open bars). Subsequently these effector cells were cocultured for 24 h with fresh non-irradiated LN-308 cells in the absence of Compound No. 79. The release of IFN-γ (B), TNF-α (C) or IL-10 (D) was assessed by Elispot assay. Data are expressed as cytokine-producing cells per 5×10⁵ effector cells (n=3, *p<0.05, **p<0.01, t-test, effect of Compound No. 79). E, F. Polyclonal NK cell cultures were exposed to TGF-β₁ (5 ng/ml) (E) or diluted (1:4) glioma cell SN (F), without or with Compound No. 79 (1 μM), for 48 h and subsequently used as effectors in ⁵¹Cr release assays using LN-308 cells as targets. Compound No. 79 alone or TGF-β antibody alone had no effect on NK cell activity in these assays (data not shown).

FIG. 4. Compound No. 79 inhibits the growth of syngeneic SMA-560 experimental gliomas in vivo and promotes immune activation. A. VM/Dk mice received an intracranial injection of 5×10³ SMA-560 cells. Three days later treatment with Compound No. 79 was initiated, and survival was monitored. B. The animals were treated as in A, but were sacrificed on day 10 to obtain splenocytes. IFN-γ release at 24 h was assessed by Elispot. Data are expressed as cytokine-producing cells per 10⁶ effector cells. C. The splenocytes were stimulated with IL-2 for 10 days to generate LAK cells. Their lytic activity was measured by ⁵¹Cr release using SMA-560 as target cells (vehicle, open squares; Compound No. 79, filled squares) (*p<0.05, t-test).

### DETAILED DESCRIPTION OF THE PREFERRED EMBODIMENT

A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application.

As used herein, the term “malignant glioma” is used in the broadest sense and refers to a brain tumor that begins in the glial cells, or supportive cells, in the brain. Without limitation, the term specifically includes astrocytomas, ependymomas, oligodendrogliomas, mixed gliomas, oligodendrogliomas, and optic nerve gliomas.

The terms “glioblastoma,” “glioblastoma multiforrne,” and “Grade IV astrocytoma,” are used herein interchangeably and in the broadest sense, to describe an aggressive form of malignant gliomas that is the most common form of brain tumor, as well as conditions characterized by or associated with such tumors. Thus, glioblastomas, such as highly cellular astrocytic tumors are typically characterized by nuclear and cellular pleomorphisms, high vascular proliferation, high mitotic figures, optionally with necrosis, microscopically infiltrative lesions, a high labeling index and other such diagnostic criteria.

As used herein, any reference to “reversing the TGF-β-mediated effects” on malignant gliomas, such as, for example, glioblastomas, means a partial or complete reversal of the effect of TGF-β on a glioma cell line, or an in vivo glioma tumor, or on the expression of a gene or protein associated with the malignant glioma (e.g. glioblastoma) relative to a normal sample of central nervous system cells of astrocytic or oligodendrocytic lineage, whichever is applicable. It is emphasized that total reversal (e.g. total return to the normal expression level) is not required, although is advantageous, under this definition.

“Treatment” is an intervention performed with the intention of preventing the development or altering the pathology of a disorder. Accordingly, “treatment” refers to both therapeutic treatment and prophylactic or preventative measures. Those in need of treatment include those already with the disorder as well as those in which the disorder is to be prevented. In tumor (e.g. glioma or glioblastoma) treatment, a therapeutic agent may directly decrease the pathology of tumor cells, or render the tumor cells more susceptible to treatment by other therapeutic agents, e.g. radiation and/or chemotherapy. Thus, treatment includes, without limitation, (1) inhibition, to some extent, of tumor growth, including slowing down and complete growth arrest; (2) reduction in the number of tumor cells; (3) reduction in tumor size; (4) inhibition (i.e., reduction, slowing down or complete stopping) of tumor cell infiltration into adjacent peripheral organs and/or tissues; (5) inhibition (i.e. reduction, slowing down or complete stopping) of metastasis; (6) enhancement of anti-tumor inmiune response, which may, but does not have to, result in the regression or rejection of the tumor; (7) relief, to some extent, of one or more symptoms associated with the tumor; relief of systemic immune suppression due to tumor derived circulating TGF-β, (8) increase in the length of survival following treatment; and/or (9) decreased mortality at a given point of time following treatment.

The “pathology” of cancer, including malignant gliomas, such as glioblastomas, includes all phenomena that compromise the well-being of the patient. This includes, without limitation, abnormal uncontrollable cell growth, metastasis, interference with the normal functioning of neighboring cells, release of cytokines (e.g. TGF-β) or other. secretory products at abnormal levels, suppression or aggravation of inflammatory or immunological response, neoplasia, premalignancy, malignancy, invasion of surrounding or distant tissues or organs.

A “therapeutically effective amount”, in reference to the treatment of a glioblastoma, e.g. when inhibitors of the present invention are used, refers to an amount capable of invoking one or more of the effects listed under the definition of “treatment” above.

The terms “specifically binding,” “binds specifically,” “specific binding,” and grammatical equivalents thereof, in the context of the type 1 TGF-β receptor, are used to refer to binding to the type 1 TGF-β receptor (TGFβ-R1) with a higher affinity than to any other polypeptide, including TGFβ-R2 and p38. Typically, specific binding means binding to a unique epitope within TGFβ-R1. The binding must occur with an affinity to effectively inhibit TGF-β signaling through TGFβ-R1. Similar definitions apply to “specific binding” to other targets.

The term “polynucleotide,” when used in singular or plural, generally refers to any polyribonucleotide or polydeoxribonucleotide, which may be unmodified RNA or DNA or modified RNA or DNA. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes DNAs and RNAs that contain one or more modified bases. Thus, DNAs or RNAs with backbones modified for stability or for other reasons are “polynucleotides” as that term is intended herein. Moreover, DNAs or RNAs comprising unusual bases, such as inosine, or modified bases, such as tritiated bases, are included within the term “polynucleotides” as defined herein. In general, the term “polynucleotide” embraces all chemically, enzymatically and/or metabolically modified forms of unmodified polynucleotides, as well as the chemical forms of DNA and RNA characteristic of viruses and cells, including simple and complex cells.

The term “oligonucleotide” refers to a relatively short polynucleotide, including, without limitation, single-stranded deoxyribonucleotides, single- or double-stranded ribonucleotides, RNA:DNA hybrids and double-stranded DNAs. Oligonucleotides, such as single-stranded DNA probe oligonucleotides, are often synthesized by chemical methods, for example using automated oligonucleotide synthesizers that are commercially available. However, oligonucleotides can be made by a variety of other methods, including in vitro recombinant DNA-mediated techniques and by expression of DNAs in cells and organisms.

The terms “differentially expressed gene,” “differential gene expression” and their synonyms, which are used interchangeably, refer to a gene whose expression is activated to a higher or lower level in a test sample relative to its expression in a normal or control sample. For the purpose of this invention, “differential gene expression” is considered to be present when there is at least an about 2.5-fold, preferably at least about 4-fold, more preferably at least about 6-fold, most preferably at least about 10-fold difference between the expression of a given gene in normal and test samples.

The term “inhibitor” as used herein refers to a molecule, e.g. a nonpeptide small molecule, specifically binding to a TGFβ-R1 receptor having the ability to inhibit a biological function of a native TGF-β molecule. Accordingly, the term “inhibitor” is defined in the context of the biological role of TGF-β and its receptors.

The term “preferentially inhibit” as used herein means that the inhibitory effect on the target that is “preferentially inhibited” is significantly greater than on any other target. Thus, in the context of preferential inhibition of TGF-β-R1 kinase relative to the p38 kinase, the term means that the inhibitor inhibits biological activities, e.g. metastatic activities of the tumor, proliferation of the tumor, necrosis, mediated by the TGF-β-R1 kinase significantly more than biological activities mediated by the p38 kinase. The difference in the degree of inhibition, in favor of the preferentially inhibited receptor, might vary, but generally is at least about two-fold, more preferably at least about five-fold, even more preferably at least about ten-fold.

The term “mammal” for purposes of treatment refers to any animal classified as a mammal, including humans, higher primates, domestic and farm animals, and zoo, sports, or pet animals, such as dogs, cats, cattle, horses, sheep, pigs, goats, rabbits, etc. Preferably, the mammal is human.

“Intracranial” means within the cranium or at or near the dorsal end of the spinal cord and includes the medulla, brain stem, pons, cerebellum and cerebrum.

Administration “in combination with” one or more further therapeutic treatments like surgery or radiation or other agents includes simultaneous (concurrent) and consecutive administration in any order. One of the preferred orders is surgery followed by radiation then chemotherapy. Chemotherapy includes combination chemotherapy; and single-agent cytotoxic chemotherapy with for example intravenous lomustine or platinums, oral carmustine, nitrosoureas; bischloroethylnitrosourea (BCNU); temozolomide or procarbazine, CCNU, vincristine (PCV); radiation sensitizing drugs). Radiation therapy includes reirradiation and or post-surgical irradiation, radiosurgery with a gamma knife or linear accelerators, low dose rate permanent-seed brachytherapy, high dose rate stereostatic brachytherapy.

In a particular aspect of.the invention, the TGF-β-R1 kinase inhibitors of the invention may be combined with other inhibitors of IGF-β including, for example, antisense strategies (Fakhrai et al., Proc. Natl. Acad. Sci. USA, 93:2909-2914 (1996)), inhibitors of TGF-β-processing proteases of the furin family, and other drugs, such as transilast (Platten et al., Int. J. Cancer, 93:53-61 (2001)). The invention further includes combination treatment with the TGFβ-R1 inhibitors of the present invention and inhibitors of other enzymes including tyrosine kinases, farnesyltransferases, and matrix metallopreteinases. Examples of such inhibitors include, but are not limited to, marimastat which is a metalloproteinase inhibitor.

As used herein, a “noninterfering substituent” is a substituent which leaves the ability of the compound as described in the formulas provided herein to inhibit TGF-β activity qualitatively intact. Thus, the substituent may alter the degree of inhibition. However, as long as the compound retains the ability to inhibit TGF-β activity, the substituent will be classified as “noninterfering.” Preferably, a “noninterfering substituent” is one whose presence does not substantially destroy the TGF-β inhibiting ability of a compound

As used herein, “hydrocarbyl residue” refers to a residue which contains only carbon and hydrogen. The residue may be aliphatic or aromatic, straight-chain, cyclic, branched, saturated or unsaturated. The hydrocarbyl residue, when indicated, may contain heteroatoms over and above the carbon and hydrogen members of the substituent residue. Thus, when specifically noted as containing such heteroatoms, the hydrocarbyl residue may also contain carbonyl groups, amino groups, hydroxyl groups and the like, or contain heteroatoms within the “backbone” of the hydrocarbyl residue.

As used herein, the term “alkyl,” “alkenyl” and “alkynyl” include straight- and branched-chain and cyclic monovalent substituents. Examples include methyl, ethyl, isobutyl, cyclohexyl, cyclopentylethyl, 2-propenyl, 3-butynyl, and the like. Typically, the alkyl, alkenyl and alkynyl substituents contain 1-10C (alkyl) or 2-10C (alkenyl or alkynyl). Preferably they contain 1-6C (alkyl) or 2-6C (alkenyl or alkynyl). Heteroalkyl, heteroalkenyl and heteroalkynyl are similarly defined but may contain 1-2 O, S or N heteroatoms or combinations thereof within the backbone residue.

As used herein, “acyl” encompasses the definitions of alkyl, alkenyl, alkynyl and the related hetero-forms which are coupled to an additional residue through a carbonyl group.

“Aromatic” moiety or “aryl” moiety refers to a monocyclic or fused bicyclic moiety such as phenyl or naphthyl; “heteroaromatic” also refers to monocyclic or fused bicyclic ring systems containing one or more heteroatoms selected from O, S and N. The inclusion of a heteroatom permits inclusion of 5-membered rings as well as 6-membered rings. Thus, typical aromatic systems include pyridyl, pyrimidyl, indolyl, benzimidazolyl, benzotriazolyl, isoquinolyl, quinolyl, benzothiazolyl, benzofuranyl, thienyl, furyl, pyrrolyl, thiazolyl, oxazolyl, imidazolyl and the like. Any monocyclic or fused ring bicyclic system which has the characteristics of aromaticity in terms of electron distribution throughout the ring system is included in this definition. Typically, the ring systems contain 5-12 ring member atoms.

Similarly, “arylalkyl” and “heteroalkyl” refer to aromatic and heteroaromatic systems which are coupled to another residue through a carbon chain, including substituted or unsubstituted, saturated or unsaturated, carbon chains, typically of 1-6C or 1-8C, or the hetero forms thereof. These carbon chains may also include a carbonyl group, thus making them able to provide substituents as an acyl or heteroacyl moiety.

B. Modes of Carrying Out the Invention

The inhibitors of the present invention are characterized by inhibiting the biological activity of one or more members of the TGF-β pathway that are associated with the development, growth or spread of glioblastomas and other malignant gliomas. In a preferred embodiment, the inhibitors of the present invention inhibit biological responses mediated by a TGF-β receptor. In another preferred embodiment, the inhibitors of the present invention selectively inhibit biological responses mediated by the type 1 TGF-β receptor, in particular matrix production, without affecting the type 2 TGF-β receptor-mediated cell proliferation.

In yet another preferred embodiment, the compounds of the present invention preferentially inhibit TGF-β R1 kinase relative to p38 kinase.

Compounds of the Invention

The present invention, at least in part, is based on the surprising discovery that gliomas, including glioblastomas, can be treated by inhibiting the biological finction of one or more members of the TGF-β signaling pathway. Inhibitors of the present invention include, without limitation, small organic molecules, peptides, polypeptides (including antibodies and antibody fragments), antisense polynucleotides, oligonucleotide decoy. molecules, and the like. In a specific embodiment, the inhibitors of the present invention are small organic molecules (non-peptide small molecules), generally less than about 1,000 daltons in size. Preferred non-peptide small molecules have molecular weights of less than about 750, daltons, more preferably less than about 500 daltons, and even more preferably less than about 300 daltons.

In a preferred embodiment, the compounds are of the formula 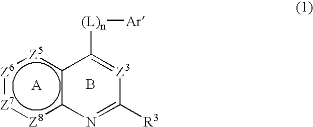
or the pharmaceutically acceptable salts thereof
wherein R³ is a noninterfering substituent;
each Z is CR² or N, wherein no more than two Z positions in ring A are N, and wherein two adjacent Z positions in ring A cannot be N;
each R² is independently a noninterfering substituent;
L is a linker;
n is 0 or 1; and
Ar′ is the residue of a cyclic aliphatic, cyclic heteroaliphatic, aromatic or heteroaromatic moiety optionally substituted with 1-3 noninterfering substituents.

In a more preferred embodiment, the small organic molecules herein are derivatives of quinazoline and related compounds containing mandatory substituents at positions corresponding to the 2- and 4-positions of quinazoline. In general, a quinazoline nucleus is preferred, although alternatives within the scope of the invention are also illustrated below. Preferred embodiments for Z³ are N and CH; preferred embodiments for Z⁵-Z⁸ are CR². However, each of Z⁵-Z⁸ can also be N, with the proviso noted above. Thus, with respect to the basic quinazoline type ring system, preferred embodiments include quinazoline per se, and embodiments wherein all of Z⁵-Z⁸ as well as Z³ are either N or CH. Also preferred are those embodiments wherein Z³ is N, and either Z⁵ or Z⁸ or both Z⁵ and Z⁸ are N and Z⁶ and Z⁷ are CH or CR². Where R² is other than H, it is preferred that CR² occur at positions 6 and/or 7. Thus, by way of example, quinazoline derivatives within the scope of the invention include compounds comprising a quinazoline nucleus, having an aromatic ring attached in position 2 as a non-interfering substituent (R³), which may be further substituted.

With respect to the substituent at the positions corresponding to the 4-position of quinazoline, LAr′, L is present or absent and is a linker which spaces the substituent Ar′ from ring B at a distance of 2-8 Å, preferably 2-6 Å, more preferably 2-4 Å. The distance is measured from the ring carbon in ring B to which one valence of L is attached to the atom of the Ar′ cyclic moiety to which the other valence of the linker is attached. The Ar′ moiety may also be coupled directly to ring B (i.e., when n is 0). Typical, but nonlimiting, embodiments of L are of the formula S(CR²₂)ₘ, —NR¹SO₂(CR²₂)₁, NR¹(CR²₂)ₘ, NR¹CO(CR²₂)₁, O(CR²₂)ₘ, OCO(CR²₂)₁, and 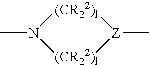
wherein Z is N or CH and wherein m is 0-4 and 1 is 0-3, preferably 1-3 and 1-2, respectively. L preferably provides —NR¹— coupled directly to ring B. A preferred embodiment of R¹ is H, but R¹ may also be acyl, alkyl, arylacyl or arylalkyl where the aryl moiety may be substituted by 1-3 groups such as alkyl, alkenyl, alkynyl, acyl, aryl, alkylaryl, aroyl, N-aryl, NH-alkylaryl, NH-aroyl, halo, OR, NR₂, SR, —SOR, —NRSOR, —NRSO₂R, —SO₂R, —OCOR, —NRCOR, —NRCONR₂, —NRCOOR, —OCONR₂, —RCO, —COOR, —SO₃R, —CONR₂, SO₂NR₂, CN, CF₃, and NO₂, wherein each R is independently H or alkyl (1-4C), preferably the substituents are alkyl (1-6C), OR, SR or NR₂ wherein R is H or lower alkyl (1-4C). More preferably, R¹ is H or alkyl (1-6C). Any aryl groups contained in the substituents may further be substituted by for example alkyl, alkenyl, alkynyl, halo, OR, NR₂, SR, —SOR, —SO₂R, —OCOR, —NRCOR, —NRCONR₂, —NRCOOR, —OCONR₂, —RCO, —COOR, SO₂R, NRSOR, NRSO₂R, —SO₃R, —CONR₂, SO₂NR₂, CN, CF₃, or NO₂, wherein each R is independently H or alkyl (1-4C).

Ar′ is aryl, heteroaryl, including 6-5 fused heteroaryl, cycloaliphatic or cycloheteroaliphatic. Preferably Ar′ is phenyl, 2-, 3- or 4-pyridyl, indolyl, 2- or 4-pyrimidyl, benzimidazolyl, indolyl, preferably each optionally substituted with a group selected from the group consisting of optionally substituted alkyl, alkenyl, alkynyl, aryl, N-aryl, NH-aroyl, halo, OR, NR₂, SR, —OOCR, —NROCR, RCO, —COOR, —CONR₂, SO₂NR₂, CN, CF₃, and NO₂, wherein each R is independently H or alkyl (1-4C).

Ar′ is more preferably indolyl, 6-pyrimidyl, 3- or 4-pyridyl, or optionally substituted phenyl.

For embodiments wherein Ar′ is optionally substituted phenyl, substituents include, without limitation, alkyl, alkenyl, alkynyl, aryl, alkylaryl, aroyl, N-aryl, NH-alkylaryl, NH-aroyl, halo, OR, NR₂, SR, —SOR, —SO₂R, —OCOR, —NRCOR, —NRCONR₂, —NRCOOR, —OCONR₂, RCO, —COOR, —SO₃R, —CONR₂, SO₂NR₂, CN, CF₃, and NO₂, wherein each R is independently H or alkyl (1-4C). Preferred substituents include halo, OR, SR, and NR₂ wherein R is H or methyl or ethyl. These substituents may occupy all five positions of the phenyl ring, preferably 1-2 positions, preferably one position. Embodiments of Ar′ include substituted or unsubstituted phenyl, 2-, 3-, or 4-pyridyl, 2-, 4- or 6-pyrimidyl, indolyl, isoquinolyl, quinolyl, benzimidazolyl, benzotriazolyl, benzothiazolyl, benzofuranyl, pyridyl, thienyl, furyl, pyrrolyl, thiazolyl, oxazolyl, imidazolyl, and morpholinyl. Particularly preferred as an embodiment of Ar′ is 3- or 4-pyridyl, especially 4-pyridyl in unsubstituted form.

Any of the aryl moieties, especially the phenyl moieties, may also comprise two substituents which, when taken together, form a 5-7 membered carbocyclic or heterocyclic aliphatic ring.

Thus, preferred embodiments of the substituents at the position of ring B corresponding to 4-position of the quinazoline include 2-(4-pyridyl)ethylamino; 4-pyridylamino; 3-pyridylamino; 2-pyridylamino; 4-indolylamino; 5-indolylamino; 3-methoxyanilinyl; 2-(2,5-difluorophenyl)ethylamino-, and the like.

R³ is generally a hydrocarbyl residue (1-20C) containing 0-5 heteroatoms selected from O, S and N. Preferably R³ is alkyl, aryl, arylalkyl, heteroalkyl, heteroaryl, or heteroarylalkyl, each unsubstituted or substituted with 1-3 substituents. The substituents are independently selected from a group that includes halo, OR, NR₂, SR, —SOR, —SO₂R, —OCOR, —NRCOR, —NRCONR₂, —NRCOOR, —OCONR₂, RCO, —COOR, —SO₃R, NRSOR, NRSO₂R, —CONR₂, SO₂NR₂, CN, CF₃, and NO₂, wherein each R is independently H or alkyl (1-4C) and with respect to any aryl or heteroaryl moiety, said group further including alkyl (1-6C) or alkenyl or alkynyl. Preferred embodiments of R³ (the substituent at position corresponding to the 2-position of the quinazoline) comprise a phenyl moiety optionally substituted with 1-2 substituents preferably halo, alkyl (1-6C), OR, NR₂, and SR wherein R is as defined above. Thus, preferred substituents at the 2-position of the quinazoline include phenyl, 2-halophenyl, e.g., 2-bromophenyl, 2-chlorophenyl, 2-fluorophenyl; 2-alkyl-phenyl, e.g., 2-methylphenyl, 2-ethylphenyl; 4-halophenyl, e.g., 4-bromophenyl, 4-chlorophenyl, 4-fluorophenyl; 5-halophenyl, e.g. 5-bromophenyl, 5-chlorophenyl, 5-fluorophenyl; 2,4- or 2,5-halophenyl, wherein the halo substituents at different positions may be identical or different, e.g. 2-fluoro 4-chlorophenyl; 2-bromo-4-chlorophenyl; 2-fluoro-5-chlorophenyl; 2-chloro-5-fluorophenyl, and the like. Other preferred embodiments of R³ comprise a cyclopentyl or cyclohexyl moiety.

As noted above, R² is a noninterfering substituent, as defined before.

Each R² is also independently a hydrocarbyl residue (1-20C) containing 0-5 heteroatoms selected from O, S and N. Preferably, R² is independently H, alkyl, alkenyl, alkynyl, acyl or hetero-forms thereof or is aryl, arylalkyl, heteroalkyl, heteroaryl, or heteroarylalkyl, each unsubstituted or substituted with 1-3 substituents selected independently from the group consisting of alkyl, alkenyl, alkynyl, aryl, alkylaryl, aroyl, N-aryl, NH-alkylaryl, NH-aroyl, halo, OR, NR₂, SR, —SOR, —SO₂R, —OCOR, —NRCOR, —NRCONR₂, —NRCOOR, NRSOR, NRSO₂R, —OCONR₂, RCO, —COOR, —SO₃R, NRSOR, NRSO₂R, —CONR₂, SO₂NR₂, CN, CF₃, and NO₂, wherein each R is independently H or alkyl (1-4C). The aryl or aroyl groups on said substituents may be further substituted by, for example, alkyl, alkenyl, alkynyl, halo, OR, NR₂, SR, —SOR, —SO₂R, —OCOR, —NRCOR, —NRCONR₂, —NRCOOR, —OCONR₂, RCO, —COOR, —SO₃R, —CONR₂, SO₂NR₂, CN, CF₃, and NO₂, wherein each R is independently H or alkyl (1-4C). More preferably the substituents on R² are selected from R⁴, halo, OR⁴, NR⁴₂, SR⁴, —OOCR⁴, —NROCR⁴, —COOR⁴, R⁴CO, —CONR⁴₂, —SO₂NR⁴₂, CN, CF₃, and NO₂, wherein each R⁴ is independently H, or optionally substituted alkyl (1-6C), or optionally substituted arylalkyl (7-12C) and wherein two R⁴ or two substituents on said alkyl or arylalkyl taken together may form a fused aliphatic ring of 5-7 members.

R2 may also, itself, be selected from the group consisting of halo, OR, NR2, SR, —SOR, —SO2R, —OCOR, —NRCOR, —NRCONR2, —NRCOOR, NRSOR, NRSO2R, —OCONR2, RCO, —COOR, —SO3R, NRSOR, NRSO2R, —CONR2, SO2NR2, CN, CF3, and NO2, wherein each R is independently H or alkyl (1-4C).

More preferred substituents represented by R2 are those as set forth with regard to the phenyl moieties contained in Ar′ or R3 as set forth above. Two adjacent CR2 taken together may form a carbocyclic or heterocyclic fused aliphatic ring of 5-7 atoms. Preferred R2 substituents are of the formula R4, —OR4, SR4 or R4NH—, especially R4NH—, wherein R4 is defined as above. Particularly preferred. are instances wherein R4 is substituted arylalkyl. Specific representatives of the compounds of formula (1) are shown in Tables 1-3 below. All compounds listed in Table 1 have a quinazoline ring system (Z3 is N), where the A ring is unsubstituted (Z5-Z8 represent CH). The substituents of the B ring are listed in the table.

TABLE 1

| Compound No. | L | Ar′ | R³ |
|---|---|---|---|
| 1 | NH | 4-pyridyl | 2-chlorophenyl |
| 2 | NH | 4-pyridyl | 2,6-dichlorophenyl |
| 3 | NH | 4-pyridyl | 2-methylphenyl |
| 4 | NH | 4-pyridyl | 2-bromophenyl |
| 5 | NH | 4-pyridyl | 2-fluorophenyl |
| 6 | NH | 4-pyridyl | 2,6-difluorophenyl |
| 7 | NH | 4-pyridyl | phenyl |
| 8 | NH | 4-pyridyl | 4-fluorophenyl |
| 9 | NH | 4-pyridyl | 4-methoxyphenyl |
| 10 | NH | 4-pyridyl | 3-fluorophenyl |
| 11* | N* | 4-pyridyl | phenyl |
| 12^{†} | N^{†} | 4-pyridyl | phenyl |
| 13 | NHCH₂ | 4-pyridyl | phenyl |
| 14 | NHCH₂ | 4-pyridyl | 4-chlorophenyl |
| 15 | NH | 4-pyridyl | phenyl |
| 16 | NHCH₂ | 4-pyridyl | phenyl |
| 17 | NHCH₂ | 4-pyridyl | phenyl |
| 18 | NHCH₂ | 4-pyridyl | phenyl |
| 19 | NHCH₂CH₂ | 4-pyridyl | phenyl |
| 20 | NH | 6-pyrimidinyl | phenyl |
| 21 | NH | 2-pyrimidinyl | phenyl |
| 22 | NH | phenyl | phenyl |
| 23 | NHCH₂ | phenyl | 3-chlorophenyl |
| 24 | NH | 3-hydroxyphenyl | phenyl |
| 25 | NH | 2-hydroxyphenyl | phenyl |
| 26 | NH | 4-hydroxyphenyl | phenyl |
| 27 | NH | 4-indolyl | phenyl |
| 28 | NH | 4-indolyl | phenyl |
| 29 | NH | 4-methoxyphenyl | phenyl |
| 30 | NH | 3-methoxyphenyl | phenyl |
| 31 | NH | 2-methoxyphenyl | phenyl |
| 32 | NH | 4-(2- | phenyl |
| | | hydroxyethyl)phenyl | |
| 33 | NH | 3-cyanophenyl | phenyl |
| 34 | NHCH₂ | 2,5-difluorophenyl | phenyl |
| 35 | NH | 4-(2-butyl)phenyl | phenyl |
| 36 | NHCH₂ | 4-dimethylaminophenyl | phenyl |
| 37 | NH | 4-pyridyl | cyclopentyl |
| 38 | NH | 4-pyridyl | phenyl |
| 39 | NHCH₂ | 4-pyridyl | phenyl |
| 40 | NH | 4-pyrimidyl | phenyl |
| 41^{∓} | N^{∓} | 4-pyridyl | phenyl |
| 42 | NH | p-aminomethylphenyl | phenyl |
| 43 | NHCH₂ | 4-aminophenyl | phenyl |
| 44 | NH | 4-pyridyl | 3-chlorophenyl |
| 45 | NH | phenyl | 4-pyridyl |
| 46 | NH | 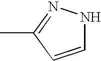 | phenyl |
| 47 | NH | 4-pyridyl | t-butyl |
| 48 | NH | 2-benzylamino-3- | phenyl |
| | | pyridyl | |
| 49 | NH | 2-benzylamino-4- | phenyl |
| | | pyridyl | |
| 50 | NH | 3-benzyloxyphenyl | phenyl |
| 51 | NH | 4-pyridyl | 3-aminophenyl |
| 52 | NH | 4-pyridyl | 4-pyridyl |
| 53 | NH | 4-pyridyl | 2-naphthyl |
| 54 | 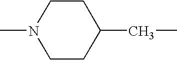 | 4-pyridyl | phenyl |
| 55 | 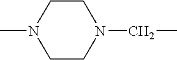 | phenyl | phenyl |
| 56 | 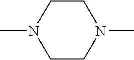 | 2-pyridyl | phenyl |
| 57 | NHCH₂CH₂ | 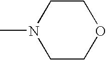 | phenyl |
| 58 | not present | 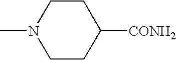 | phenyl |
| 59 | not present | 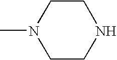 | phenyl |
| 60 | NH | 4-pyridyl | cyclopropyl |
| 61 | NH | 4-pyridyl | 2-trifluoromethyl |
| | | | phenyl |
| 62 | NH | 4-aminophenyl | phenyl |
| 63 | NH | 4-pyridyl | cyclohexyl |
| 64 | NH | 4-methoxyphenyl | 2-fluorophenyl |
| 65 | NH | 4-methoxyphenyl | 2-fluorophenyl |
| 66 | NH | 4-pyrimidinyl | 2-fluorophenyl |
| 67 | NH | 4-amino-4-pyridyl | phenyl |
| 68 | NH | 4-pyridyl | 2- |
| | | | benzylaminophenyl |
| 69 | NH | 2-benzylaminophenyl | phenyl |
| 70 | NH | 2-benzylaminophenyl | 4-cyanophenyl |
| 71 | NH | 3′-cyano-2- | phenyl |
| | | benzylaminophenyl | |

*R¹ = 2-propyl
^{†}R¹ = 4-methoxyphenyl
^{∓}R¹ = 4-methoxybenzyl

The compounds in Table 2 contain modifications of the quinazoline nucleus shown. All of the compounds in Table 2 are embodiments of formula (1) wherein Z³ is N and Z⁶ and Z⁷ represent CH. In all cases the linker, L, is present and is NH.

TABLE 2

| Compound No. | Z⁵ | Z⁸ | Ar¹ | R³ |
|---|---|---|---|---|
| 72 | CH | N | 4-pyridyl | 2-fluorophenyl |
| 73 | CH | N | 4-pyridyl | 2-chlorophenyl |
| 74 | CH | N | 4-pyridyl | 5-chloro-2- |
| | | | | fluorphenyl |
| 75 | CH | N | 4-(3-methyl)-pyridyl | 5-chloro-2- |
| | | | | fluorphenyl |
| 76 | CH | N | 4-pyridyl | Phenyl |
| 77 | N | N | 4-pyridyl | phenyl |
| 78 | N | CH | 4-pyridyl | Phenyl |
| 79 | N | N | 4-pyridyl | 5-chloro-2- |
| | | | | fluorphenyl |
| 80 | N | N | 4-(3-methyl)-pyridyl | 5-chloro-2- |
| | | | | fluorphenyl |

Additional compounds were prepared wherein ring A contains CR² at Z⁶ or Z⁷ where R² is not H. These compounds, which are all quinazoline derivatives, wherein L is NH and Ar′ is 4-pyridyl, are shown in Table 3.

TABLE 3

| Compound No. | R³ | CR² as noted |
|---|---|---|
| 81 | 2-chlorophenyl | 6,7-dimethoxy |
| 82 | 2-fluorophenyl | 3-nitro |
| 83 | 2-fluorophenyl | 6-amino |
| 84 | 2-fluorophenyl | 7-amino |
| 85 | 2-fluorophenyl | 6-(3-methoxybenzylamino) |
| 86 | 2-fluorophenyl | 3-(4-methoxybenzylamino) |
| 87 | 2-fluorophenyl | 6-(2-isobutylamino) |
| 88 | 2-fluorophenyl | 6-(4- |
| | | methylmercaptobenzylamino) |
| 89 | 2-fluorophenyl | 6-(4-methoxybenzoyl amino) |
| 90 | 4-fluorophenyl | 7-amino |
| 91 | 4-fluorophenyl | 7-(3-methoxybenzylamino) |

Further representative structures of compounds (1) of the invention are shown below in Table 4.

TABLE 4

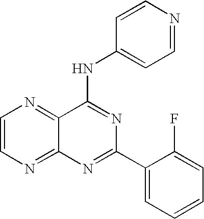

92

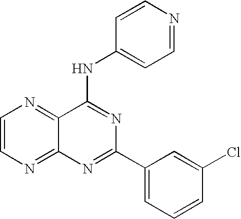

93

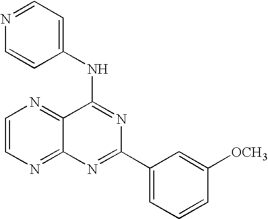

94

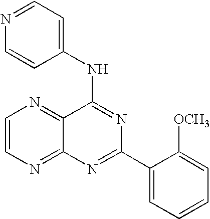

95

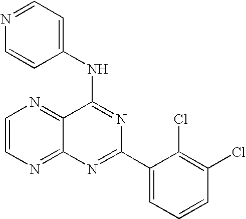

96

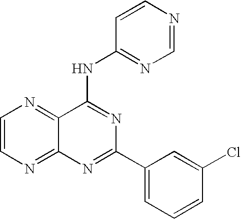

97

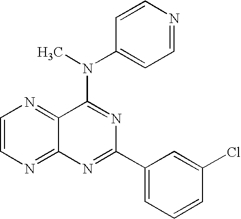

98

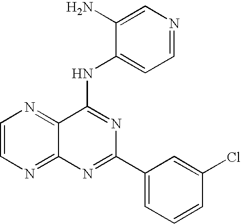

99

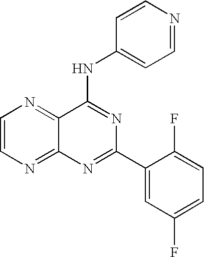

100

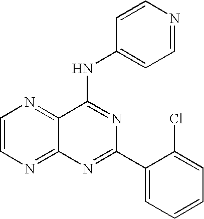

101

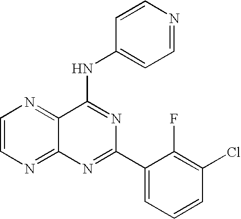

102

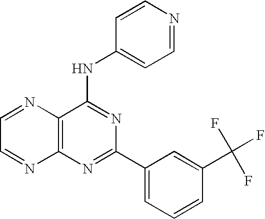

103

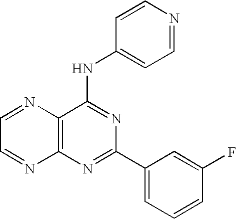

104

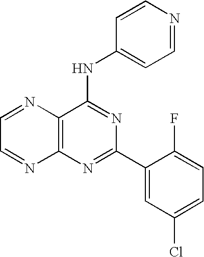

105

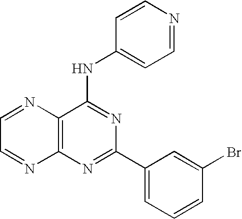

106

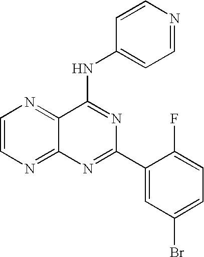

107

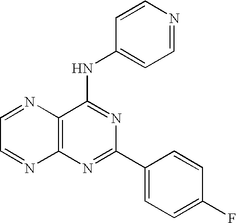

108

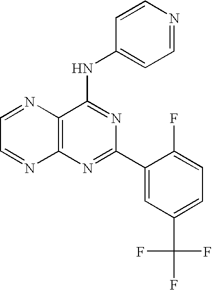

109

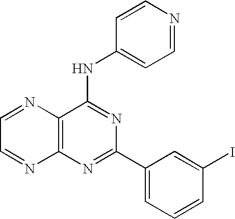

110

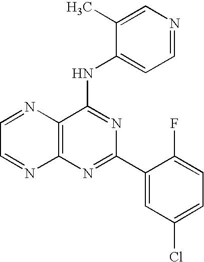

111

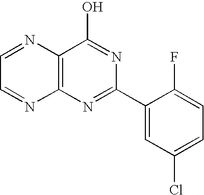

112

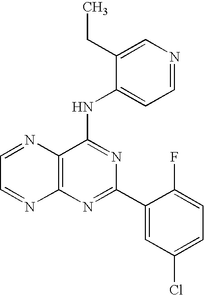

113

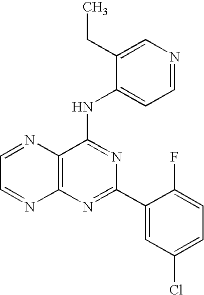

114

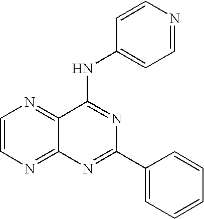

115

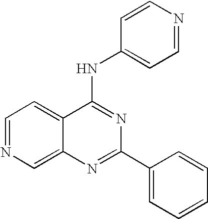

116

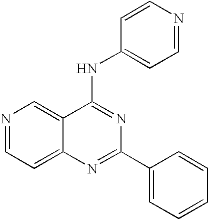

117

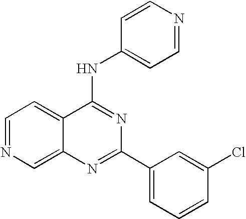

118

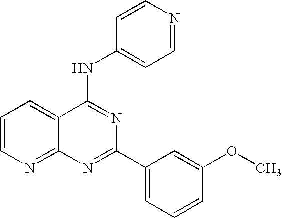

119

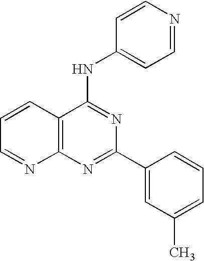

120

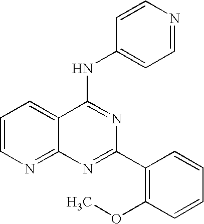

121

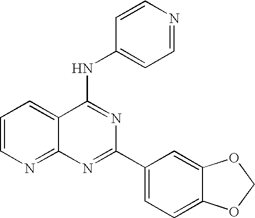

122

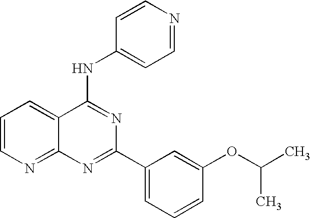

123

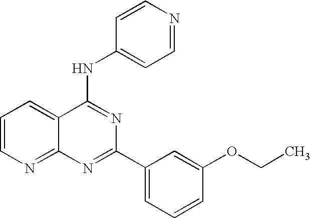

124

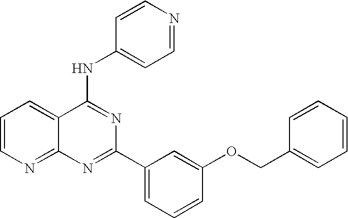

125

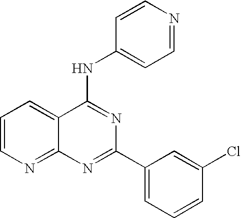

126

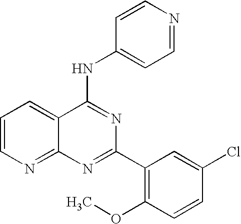

127

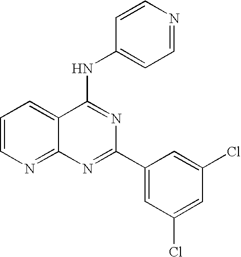

128

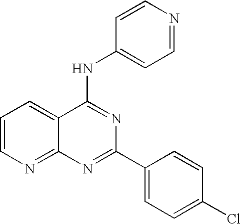

129

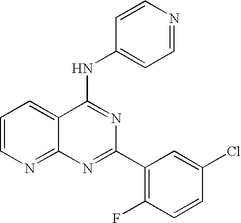

130

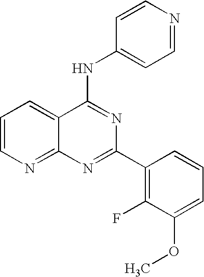

131

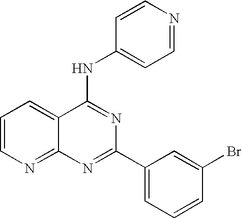

132

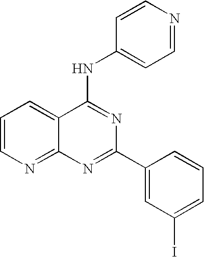

133

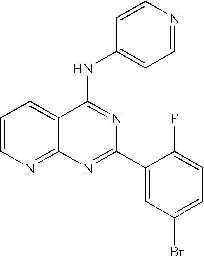

134

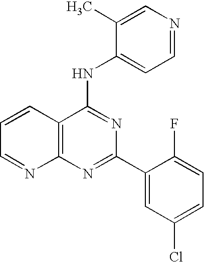

135

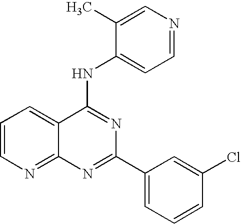

136

As apparent from the disclosure above, although many of the compounds of formula (1) useful in the methods of the present invention are quinazoline derivatives, the present invention includes the used of compounds of formula (1) having a non-quinazoline, such as, a pyridine, pyrimidine nucleus carrying substituents like those discussed above with respect to the quinazoline derivatives. Compounds of formula (1) are also disclosed in PCT Publication No. WO 00/12497, published Mar. 9, 2003, the entire disclosure of which is hereby expressly incorporated by reference.

Another group of compounds for use in the methods of the present invention is represented by the following formula (2) 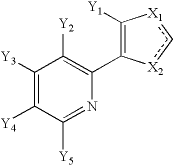
or the pharmaceutically acceptable salts or prodrug forms thereof; wherein:
Y₁ is phenyl or naphthyl optionally substituted with one or more substituents selected from halo, alkoxy(1-6 C), alkylthio(1-6 C), alkyl(1-6 C), haloalkyl (1-6C), —O—(CH₂)ₘ-Ph, —S—(CH₂)ₘ-Ph, cyano, phenyl, and CO₂R, wherein R is hydrogen or alkyl(1-6 C), and m is 0-3; or phenyl fused with a 5- or 7-membered aromatic or non-aromatic ring wherein said ring contains up to three heteroatoms, independently selected from N, O, and S:
Y₂, Y₃, Y₄, and Y₅ independently represent hydrogen, alkyl(1-6C), alkoxy(1-6 C), haloalkyl(1-6 C), halo, NH₂, NH-alkyl(1-6C), or NH(CH₂)ₙ-Ph wherein n is 0-3; or an adjacent pair of Y₂, Y₃, Y₄, and Y₅ form a fused 6-membered aromatic ring optionally containing up to 2 nitrogen atoms, said ring being optionally substituted by one or more substituents independently selected from alkyl(1-6 C), alkoxy(a-6 C), haloalkyl(1-6 C), halo, NH₂, NH-alkyl(1-6 C), or NH(CH₂)ₙ-Ph, wherein n is 0-3, and the remainder of Y₂, Y₃, Y₄, and Y₅ represent hydrogen, alkyl(1-6 C), alkoxy(l-6C), haloalkyl(1-6 C), halo, NH_{2,} NH-alkyl(1-6 C), or NH(CH₂)ₙ-Ph wherein n is 0-3; and
one of X₁ and X₂ is N and the other is NR₆, wherein R₆ is hydrogen or alkyl(1-6C).

As used in formula (2), the double bonds indicated by the dotted lined represent possible tautomeric ring forms of the compounds. Further information about compounds of formula (2) and their preparation is disclosed in WO 02/40468, published May 23, 2002, the entire disclosure of which is hereby expressly incorporated by reference.

Yet another group of compounds for use in the methods of the invention is represented by the following formula (3): 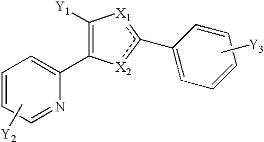
or the pharmaceutically acceptable salts or prodrug forms thereof; wherein:
Y₁ is naphthyl, anthracenyl, or phenyl optionally substituted with one or more substituents selected from the group consisting of halo, alkoxy(1-6 C), alkylthio(1-6 C), alkyl(1-6 C), —O—(CH₂)-Ph, —S—(CH₂)ₙ-Ph, cyano, phenyl, and CO₂R, wherein R is hydrogen or alkyl(1-6 C), and n is 0, 1, 2, or 3; or Y₁ represents phenyl fused with an aromatic or non-aromatic cyclic ring of 5-7 members wherein said cyclic ring optionally contains up to two heteroatoms, independently selected from N, O, and S;
Y₂ is H, NH(CH₂)ₙ-Ph or NH-alkyl(1-6 C), wherein n is 0, 1, 2, or 3;
Y₃ is CO₂H, CONH₂, CN, NO₂, alkylthio(1-6 C), —SO₂-alkyl(C1-6), alkoxy(C1-6), SONH₂, CONHOH, NH₂, CHO, CH₂NH₂, or CO₂R, wherein R is hydrogen or alkyl(1-6 C);
one of X₁ and X₂ is N or CR′, and other is NR′ or CHR′ wherein R′ is hydrogen, OH, alkyl(C-16), or cycloalkyl(C3-7); or when one of X₁ and X₂ is N or CR′ then the other may be S or O.

Further details of the compounds of formula (3) and their modes of preparation are disclosed in WO 00/61576 published Oct. 19, 2000, the entire disclosure of which is hereby expressly incorporated by reference.

In a ftrther embodiment, the TGF-β inhibitors of the present invention are represented by the following formula (4): 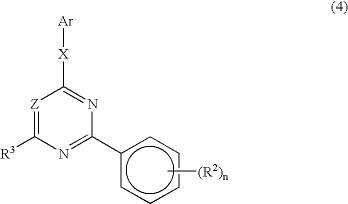
or the pharmaceutically acceptable salts or prodrug forms thereof; wherein:
Ar represents an optionally substituted aromatic or optionally substituted heteroaromatic moiety containing 5-12 ring members wherein said heteroaromatic moiety contains one or more O, S, and/or N with a proviso that the optionally substituted Ar is not 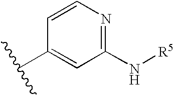
wherein R5 is H, alkyl (1-6C), alkenyl (2-6C), alkynyl (2-6C), an aromatic or heteroaromatic moiety containing 5-11 ring members;
X is NR¹, O, or S;
R¹ is H, alkyl (1-8C), alkenyl (2-8C), or alkynyl (2-8C);
Z represents N or CR⁴;
each of R³ and R⁴ is independently H, or a non-interfering substituent;
each R² is independently a non-interfering substituent; and
n is 0, 1, 2, 3, 4, or 5. In one embodiment, if n>2, and the R²'s are adjacent, they can be joined together to form a 5 to 7 membered non-aromatic, heteroaromatic, or aromatic ring containing 1 to 3 heteroatoms where each heteroatom can independently be O, N, or S.

In preferred embodiments, Ar represents an optionally substituted aromatic or optionally substituted heteroaromatic moiety containing 5-9 ring members wherein said heteroaromatic moiety contains one or more N; or
R1 is H, alkyl (1-8C), alkenyl (2-8C), or alkynyl (2-8C); or
Z represents N or CR4; wherein
R⁴ is H, alkyl (1-10C), alkenyl (2-10C), or alkynyl (2-10C), acyl (1-10C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, or —NO₂, wherein each R is independently H or alkyl (1-10C) or a halo or heteroatom-containing form of said alkyl, each of which may optionally be substituted. Preferably R⁴ is H, alkyl (1-10C), OR, SR or NR₂ wherein R is H or alkyl (1-10C) or is O-aryl; or
R³ is defined in the same manner as R⁴ and preferred forms are similar, but R³ is independently embodied; or
each R² is independently alkyl (1-8C), alkenyl (2-8C), alkynyl (2-8C), acyl (1-8C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —NRSO₂R₂, —SO₂R, —OCOR, —OSO₃R, —NRCOR, —NRCONR₂, —NRCOOR, —OCONR₂, —COOR, —SO₃R, —CONR₂, SO₂NR₂, —CN, —CF₃, or —NO₂, wherein each R is independently H or lower alkyl (1-4C). Preferably R² is halo, alkyl (1-6C), OR, SR or NR₂ wherein R is H or lower alkyl (1-4C), more preferably halo; or n is 0-3.

The optional substituents on the aromatic or heteroaromatic moiety represented by Ar include alkyl (1-10C), alkenyl (2-10C), alkynyl (2-10C), acyl (1-10C), aryl, alkylaryl, aroyl, O-aryl, O-alkylaryl, O-aroyl, NR-aryl, NR-alkylaryl, NR-aroyl, or the hetero forms of any of the foregoing, halo, OR, NR₂, SR, —SOR, —NRSOR, —NRSO₂R, —SO₂R, —OCOR, —NRCOR, —NRCONR₂, —NRCOOR, —OCONR₂, —COOR, —SO₃R, —CONR₂, —SO₂NR₂, —CN, —CF₃, and/or NO₂, wherein each R is independently H or lower alkyl (1-4C). Preferred substituents include alkyl, OR, NR₂, O-alkylaryl and NH-alkylaryl.

In general, any alkyl, alkenyl, alkynyl, acyl, or aryl group contained in a substituent may itself optionally be substituted by additional substituents. The nature of these substituents is similar to those recited with regard to the primary substituents themselves.

Representative compounds of formula (4) are listed in the following Table 5.

TABLE 5

| COMPOUND | |
|---|---|
| # | STRUCTURE |
| 137 | 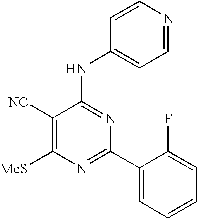 |
| 138 | 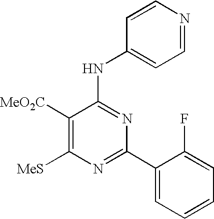 |
| 139 | 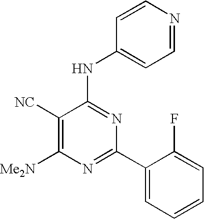 |
| 140 | 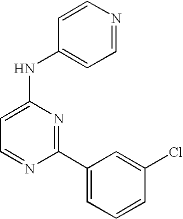 |
| 141 | 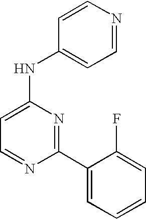 |
| 142 | 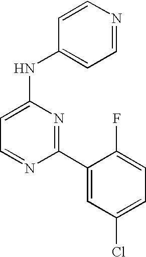 |
| 143 | 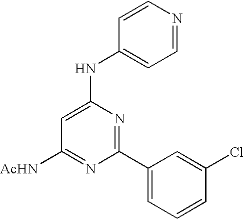 |
| 144 | 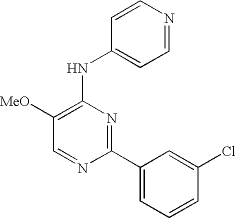 |
| 145 | 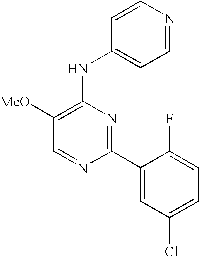 |
| 146 | 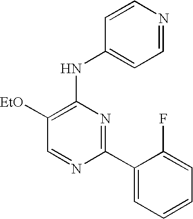 |
| 147 | 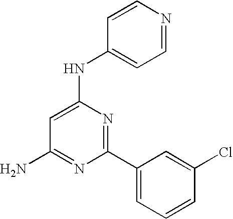 |
| 148 | 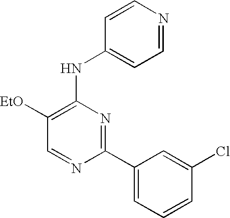 |
| 149 | 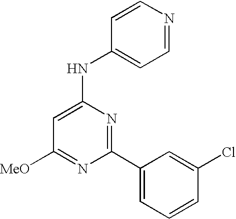 |
| 150 | 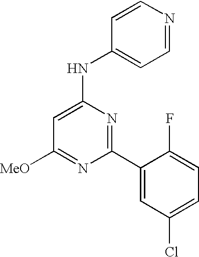 |
| 151 | 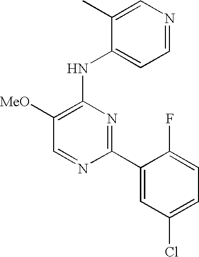 |
| 152 | 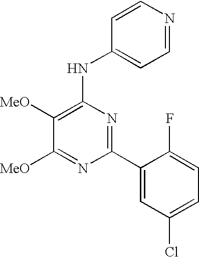 |
| 153 | 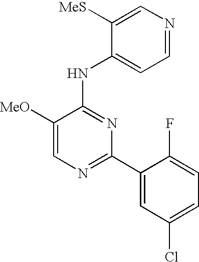 |
| 154 | 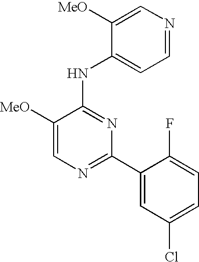 |
| 155 | 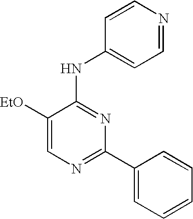 |
| 156 | 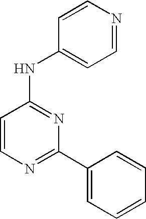 |
| 157 | 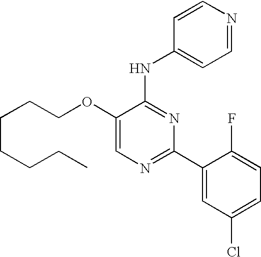 |
| 158 | 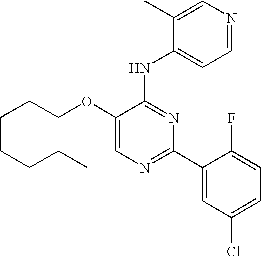 |
| 159 | 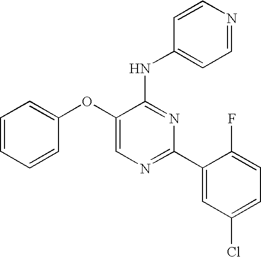 |
| 160 | 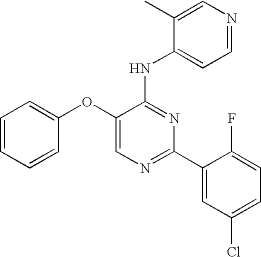 |
| 161 | 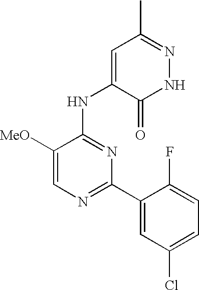 |
| 162 | 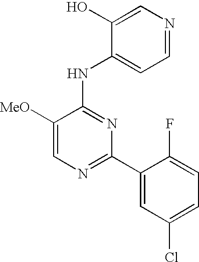 |
| 163 | 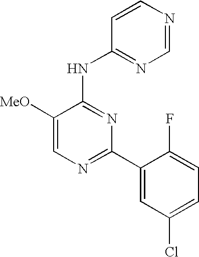 |
| 164 | 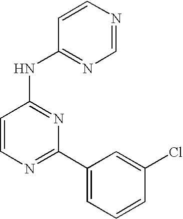 |
| 165 | 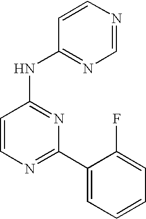 |
| 166 | 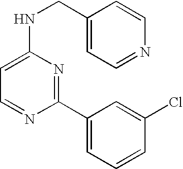 |
| 167 | 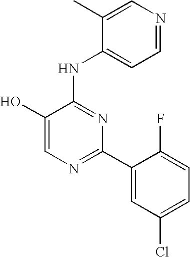 |
| 168 | 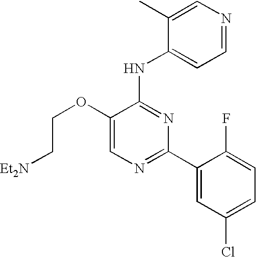 |
| 169 | 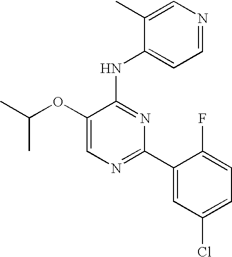 |
| 170 | 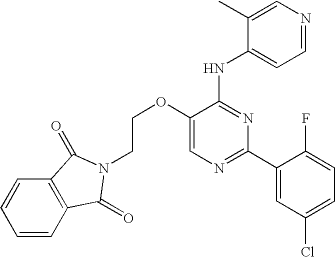 |
| 171 | 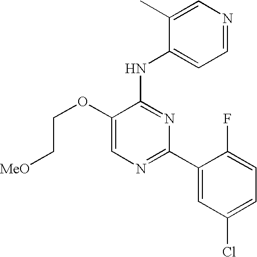 |
| 172 | 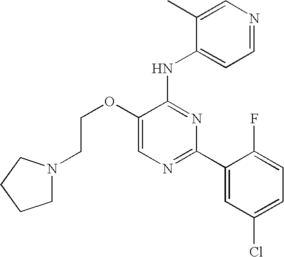 |
| 173 | 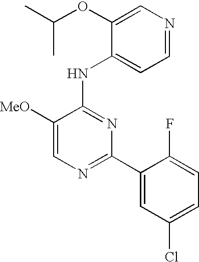 |
| 174 | 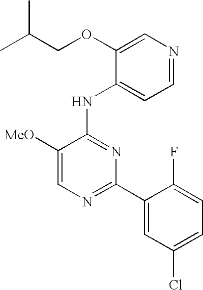 |
| 175 | 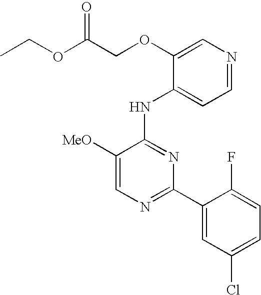 |
| 176 | 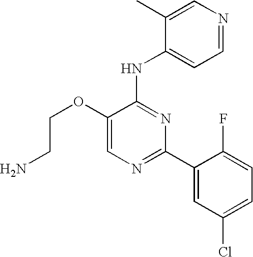 |
| 177 | 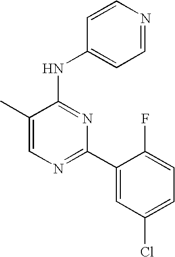 |
| 178 | 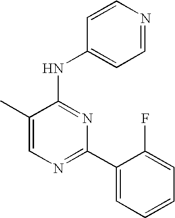 |
| 179 | 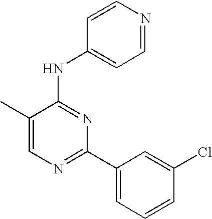 |
| 180 | 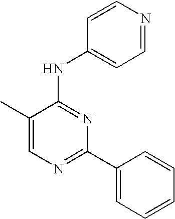 |
| 181 | 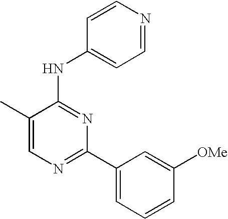 |
| 182 | 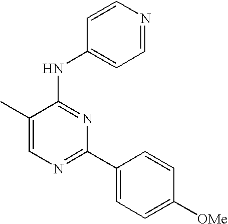 |
| 183 | 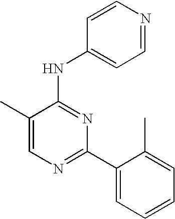 |
| 184 | 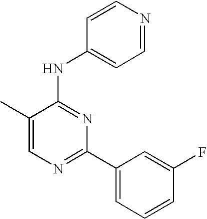 |
| 185 | 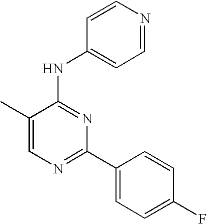 |
| 186 | 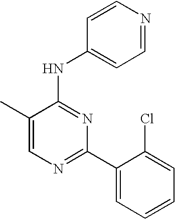 |
| 187 | 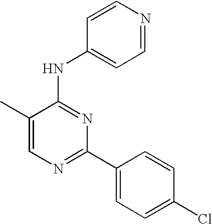 |
| 188 | 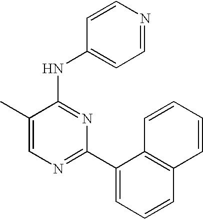 |
| 189 | 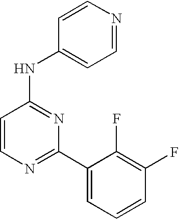 |
| 190 | 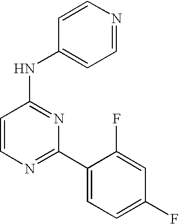 |
| 191 | 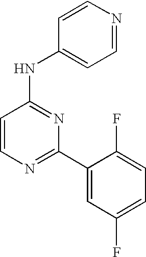 |
| 192 | 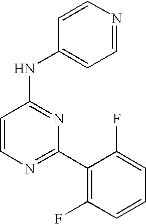 |
| 193 | 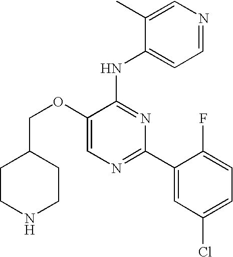 |
| 194 | 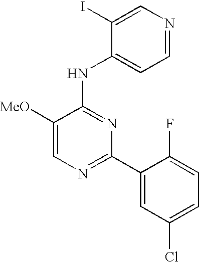 |
| 195 | 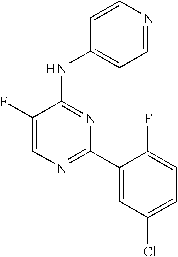 |
| 196 | 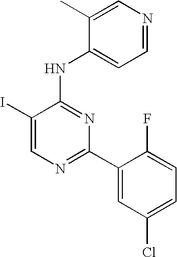 |
| 197 | 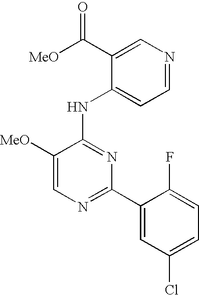 |
| 198 | 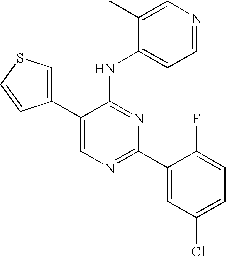 |
| 199 | 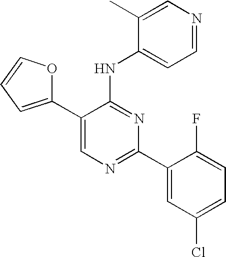 |
| 200 | 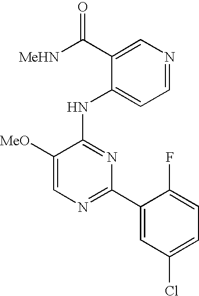 |
| 201 | 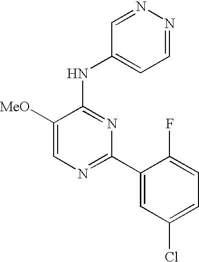 |
| 202 | 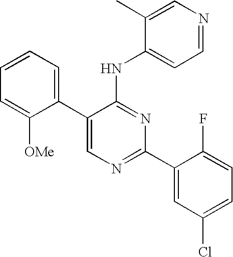 |
| 203 | 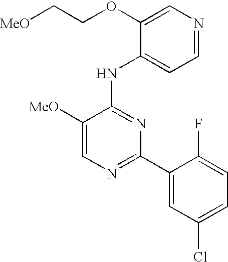 |
| 204 | 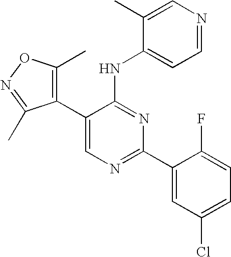 |
| 205 | 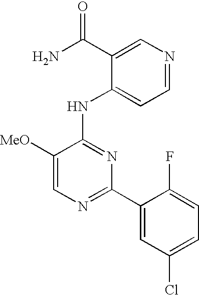 |
| 206 | 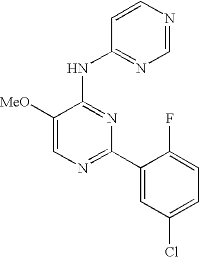 |
| 207 | 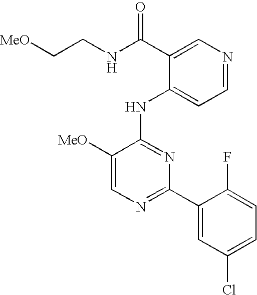 |
| 208 | 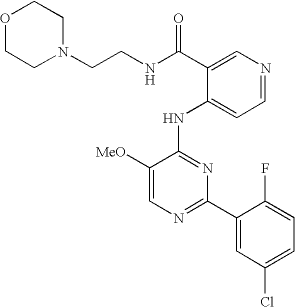 |
| 209 | 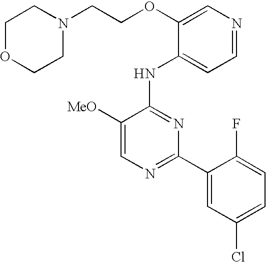 |
| 210 | 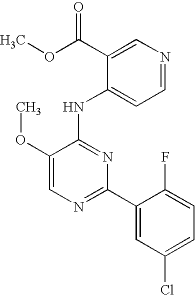 |
| 211 | 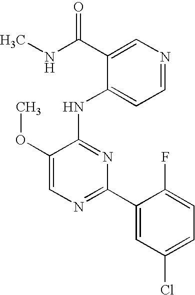 |
| 212 | 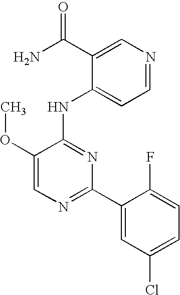 |
| 213 | 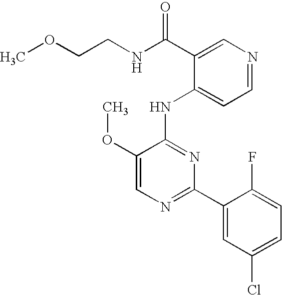 |
| 214 | 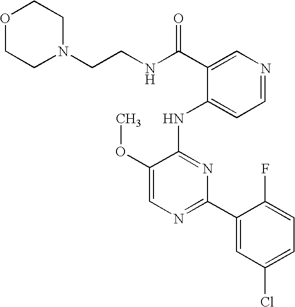 |
| 215 | 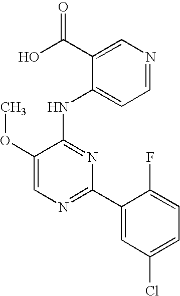 |
| 216 | 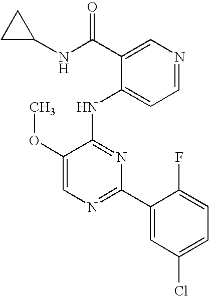 |
| 217 | 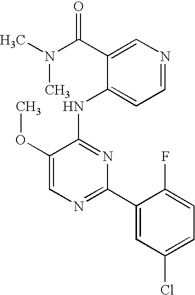 |
| 218 | 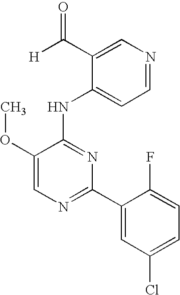 |
| 219 | 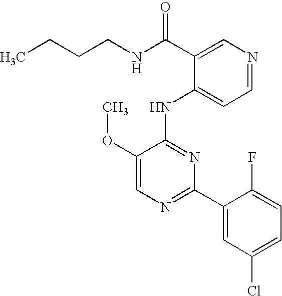 |
| 220 | 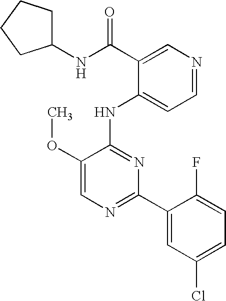 |
| 221 | 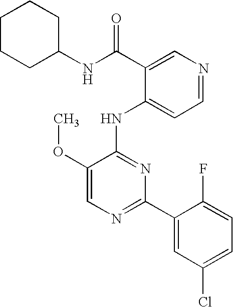 |
| 222 | 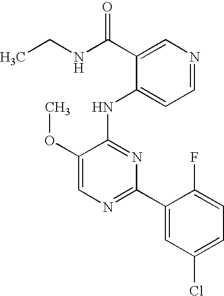 |
| 223 | 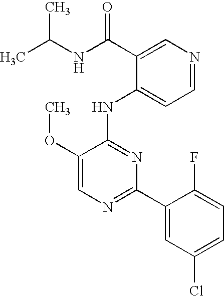 |
| 224 | 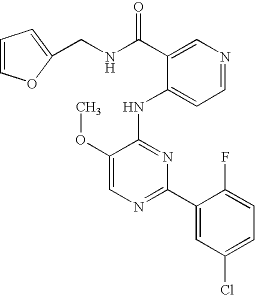 |
| 225 | 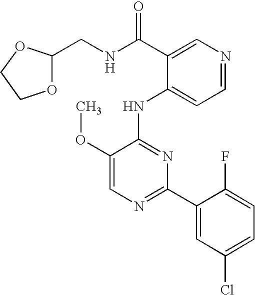 |
| 226 | 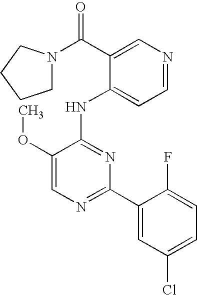 |
| 227 | 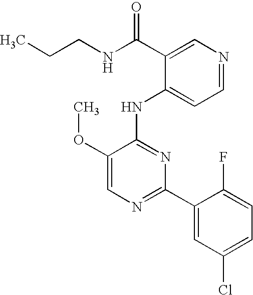 |
| 228 | 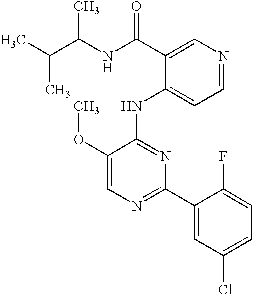 |
| 229 | 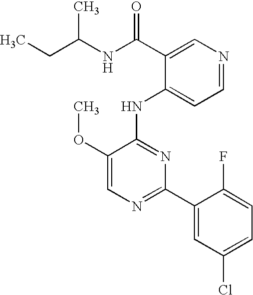 |
| 230 | 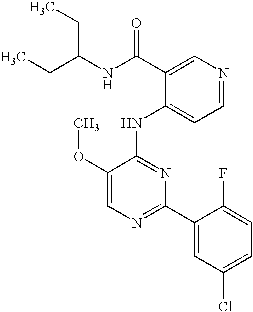 |
| 231 | 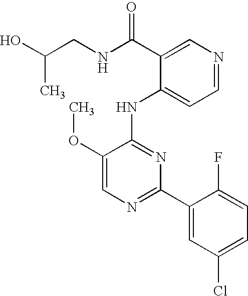 |
| 232 | 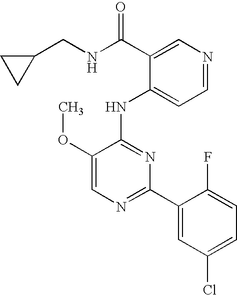 |
| 233 | 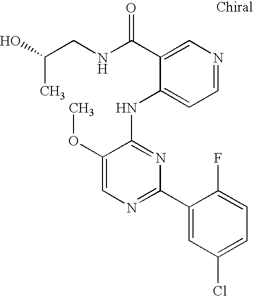 |
| 234 | 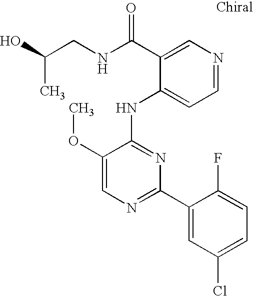 |
| 235 | 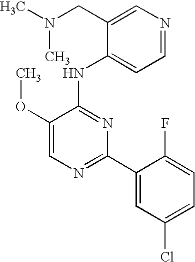 |
| 236 | 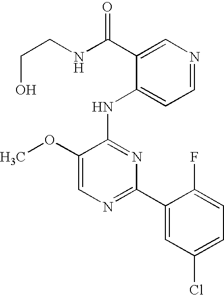 |
| 237 | 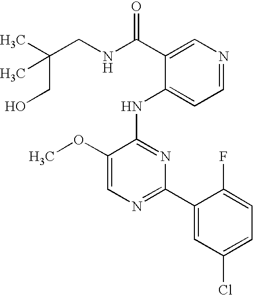 |
| 238 | 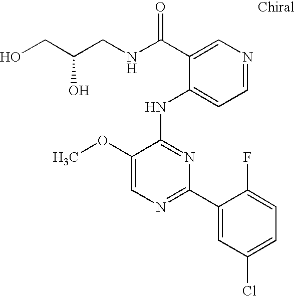 |
| 239 | 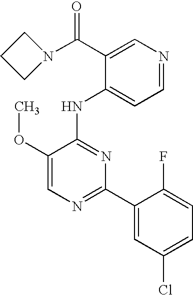 |
| 240 | 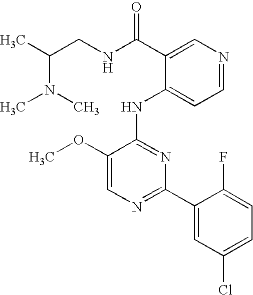 |
| 241 | 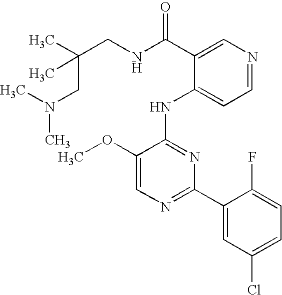 |
| 242 | 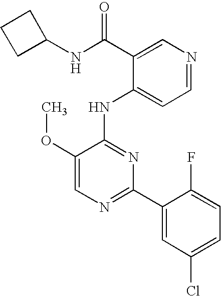 |
| 243 | 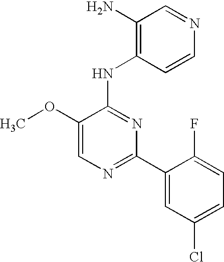 |
| 244 | 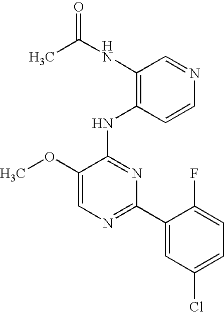 |
| 245 | 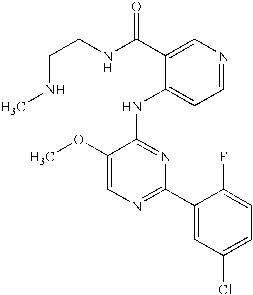 |
| 246 | 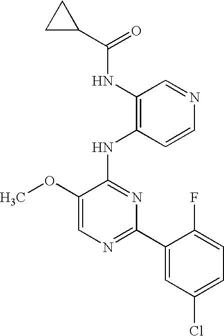 |
| 247 | 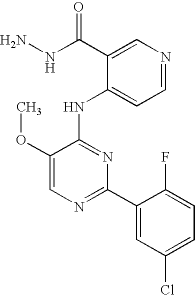 |
| 248 | 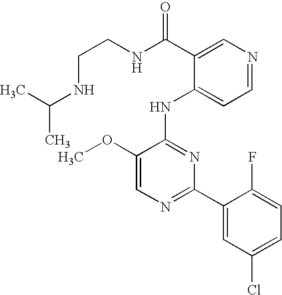 |
| 249 | 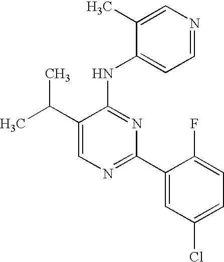 |
| 250 | 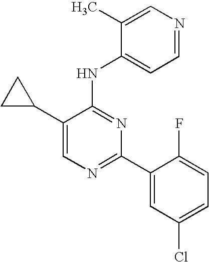 |
| 251 | 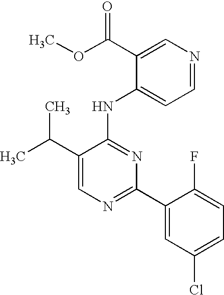 |
| 252 | 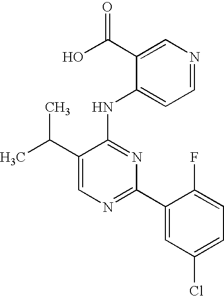 |
| 253 | 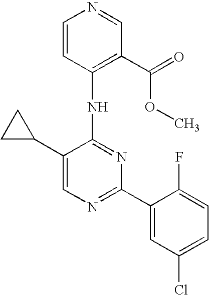 |
| 254 | 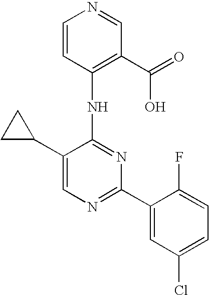 |
| 255 | 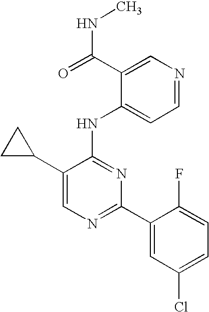 |
| 256 | 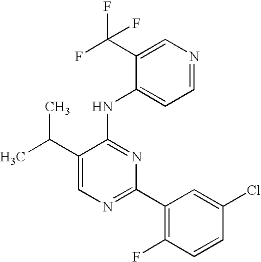 |
| 257 | 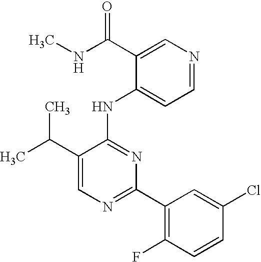 |
| 258 | 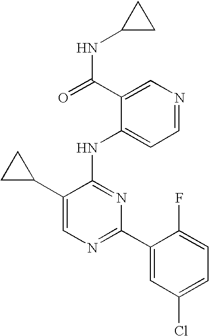 |
| 259 | 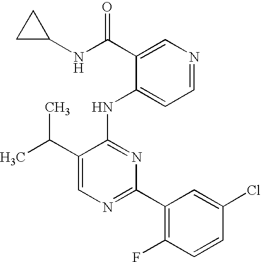 |
| 260 | 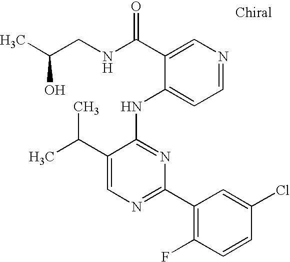 |
| 261 | 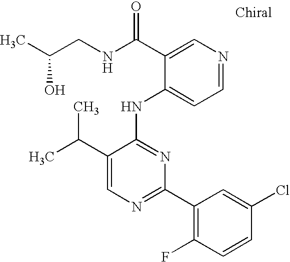 |
| 262 | 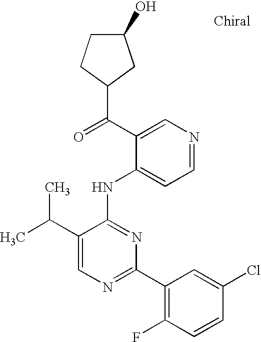 |
| 263 | 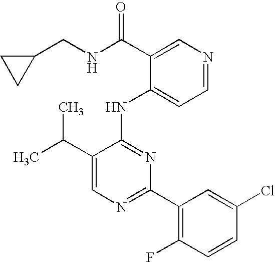 |
| 264 | 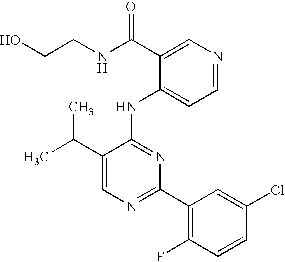 |
| 265 | 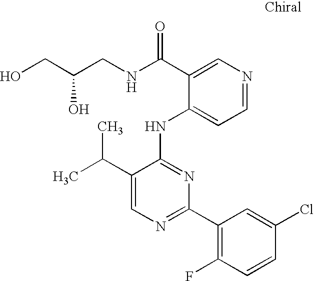 |
| 266 | 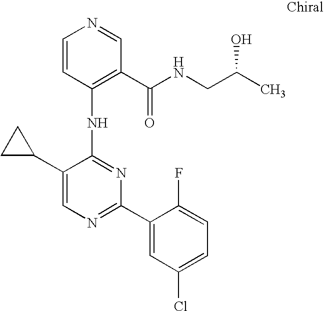 |
| 267 | 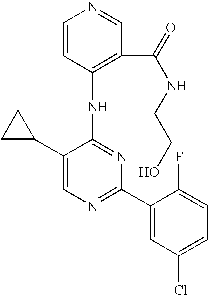 |
| 268 | 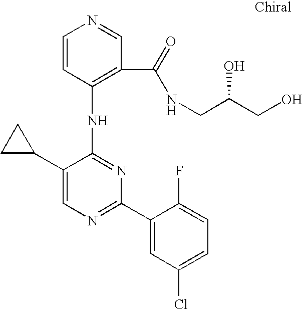 |
| 269 | 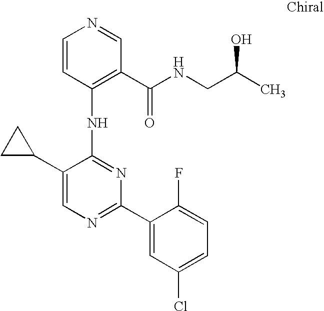 |
| 270 | 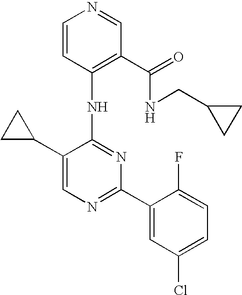 |
| 271 | 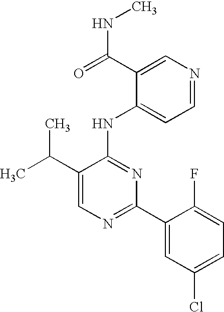 |
| 272 | 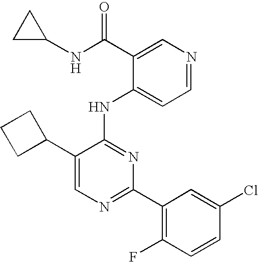 |
| 273 | 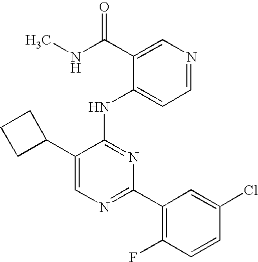 |
| 274 | 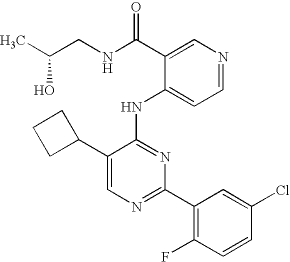 |
| 275 | 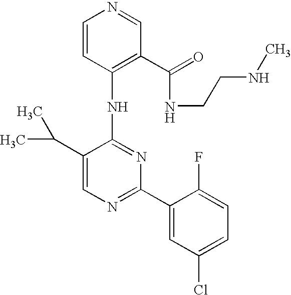 |
| 276 | 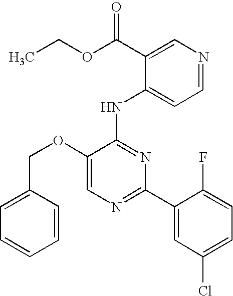 |
| 277 | 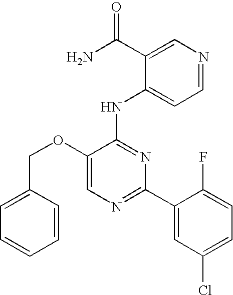 |
| 278 | 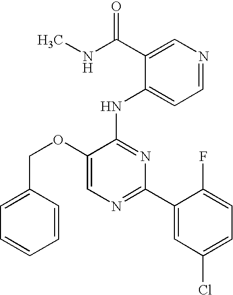 |
| 279 | 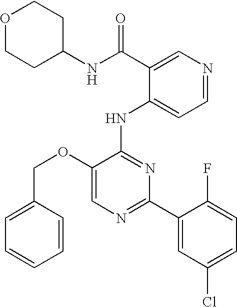 |
| 280 | 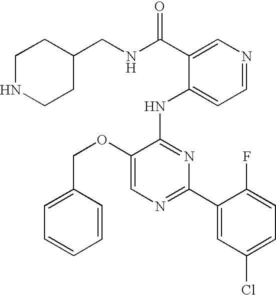 |
| 281 | 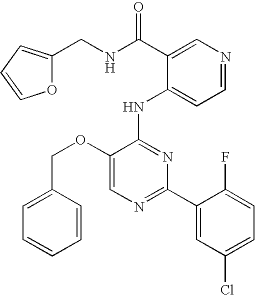 |
| 282 | 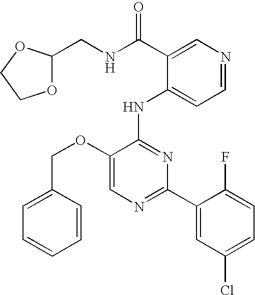 |
| 283 | 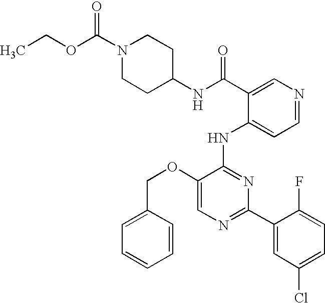 |
| 284 | 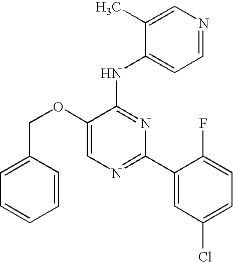 |
| 285 | 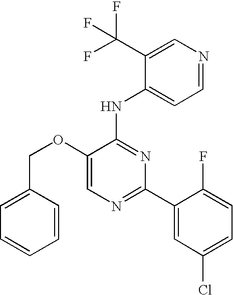 |
| 286 | 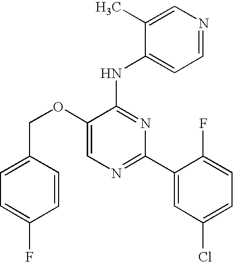 |
| 287 | 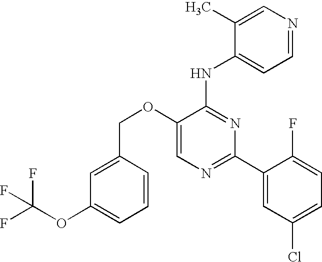 |
| 288 | 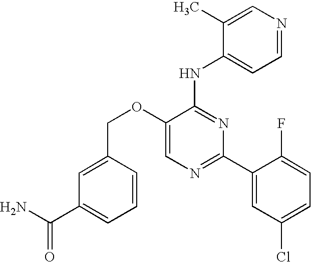 |
| 289 | 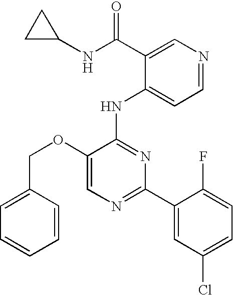 |
| 290 | 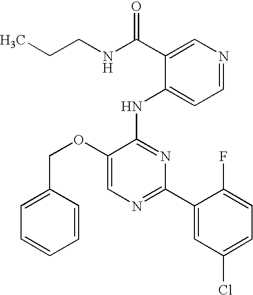 |
| 291 | 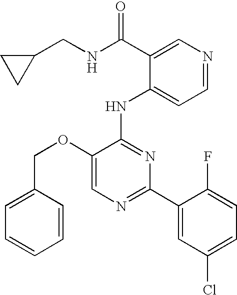 |
| 292 | 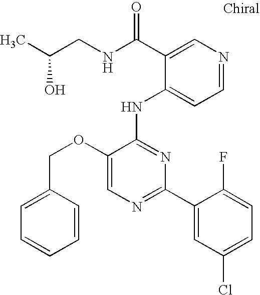 |
| 293 | 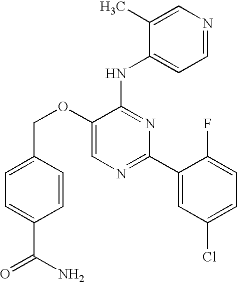 |
| 294 | 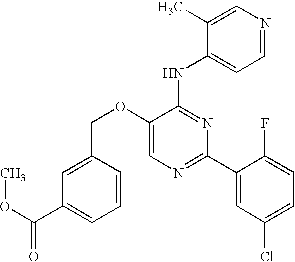 |
| 295 | 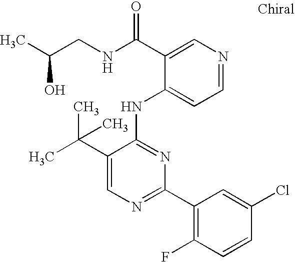 |
| 296 | 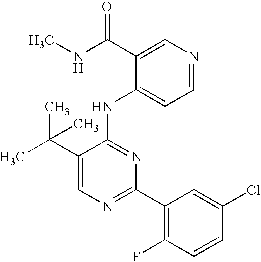 |

Further TGF-β inhibitors for use in the methods of the present invention are represented by formula (5): 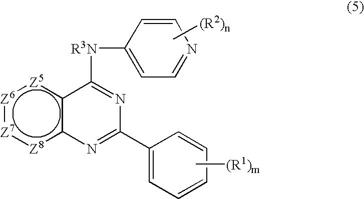
or the pharmaceutically acceptable salts thereof; wherein:
each of Z⁵, Z⁶, Z⁷ and Z⁸ is N or CH and wherein one or two Z⁵, Z⁶, Z⁷ and Z⁸ are N and wherein two adjacent Z positions cannot be N;
m and n are each independently 0-3;
R¹ is halo, alkyl, alkoxy or alkyl halide and wherein two adjacent R¹ groups may be joined to form an aliphatic heterocyclic ring of 5-6 members;
R² is a noninterfering substituent; and
R³ is H or CH₃.

The compounds of formula (5) are derivatives of quinazoline and related compounds containing mandatory substituents at positions corresponding to the 2- and 4-positions of the quinazoline. Preferably, the compounds of formula (5) include a pteridine or pyridopyrimidine nucleus. Pteridine and 8-pyrido pyrimidine nuclei are preferred. Thus, in one embodiment Z⁵ and Z⁸ are N, and Z⁶ and Z⁷ are CH. However in all cases, at least one of each of Z⁵-Z⁸ must be N. Preferred embodiments for R¹ are halo, preferably F, Cl, I or Br, most preferably Cl or F, NR₂, OH or CF₃.

The position that corresponds to the 2-position of the quinazoline contains a mandatory phenyl substituent.

The position that corresponds to the 4-position of the quinazoline contains a mandatory —NR³-4′-pyridyl substituent that may optionally contain 0-4 non-interfering substituents, namely (R²)ₙ, wherein n is 0-4. Preferably, the pyridyl group is unsubstituted, i.e., n is 0. When substituted, the pyridyl moiety is preferably substituted with an alkyl group. such as methyl or ethyl, or a halo group preferably bromo or iodo each of which are preferably substituted at the ortho position relative to the pyridyl's linkage to the quinazoline derivative nucleus. In another embodiment, n is 1, and R³ is methyl, preferably, at the 1′ or 2′ position.

The R¹ substituent(s) preferably include minimally bulky groups such as halo, lower alkyl, lower alkoxy, and lower alkyl halide groups. Preferably such groups include one or more halo, such as Cl, F, Br, and I which may be the same or different if more than two halo groups are present; alkyl halide containing 1-3 halides, preferably methyl halide and even more preferably trifluoro methyl; OH; R which is a lower alkyl, preferably C1-6, more preferably C1-3 alkyl, and even more preferably, methyl, ethyl, propyl or isopropyl, most preferably methyl; OR were R is defined as above and OR is preferably methoxy, ethoxy, isopropoxy, methyl phenyloxy. Two adjacent R groups may join to make an aliphatic or hetero aliphatic ring fused to the 2-phenyl. Preferably, if a fused ring is present it has 5 or 6 members, preferably 5 members and contains 1 or more heteroatoms such as N, S or O, and preferably O. Preferably, the fused ring is 1, 3 dioxolane fused to phenyl at the 4 and 5 position of the phenyl ring.

The R¹ group or groups that are bound to the 2-phenyl group may be bound at any available position of the phenyl ring. Preferably the R¹ group is bound at the position meta relative to the phenyl's attachment point on the quinazoline derivative nucleus. Also, in a preferred embodiment when phenyl is substituted with two groups, the groups are bound at the ortho and meta positions relative to the phenyl's attachment to the quinazoline derivative, more preferably at non-adjacent ortho and meta positions. Other embodiments include such groups at the ortho or para positions. A phenyl substituted at both meta positions or adjacent ortho and meta positions are contemplated if two groups are present. Alternatively, two groups may form a fused ring preferably attached at the meta and para positions relative to the phenyl's attachment to the quinazoline derivative. Also it is contemplated the phenyl is unsubstituted.

For compounds containing pyridopyrimidine as the nucleus, when the 6- or 7-isomers thereof are present, i.e. the nitrogen is in position 6 or 7 of pyridopyrimidine, the phenyl preferably is unsubstituted, or preferably contains one halo substituent, preferably chlorine, and preferably attached at the meta position relative to the phenyl's attachment to the pyridopyrimidine moiety.

In the compounds of formula (5), preferably, the phenyl is substituted, preferably with halo, more preferably one or two halos, and even more preferably chloro at the meta or para positions relative to the phenyl's attachment to the pyridopyrimidine moiety or dichloro at both meta positions; or more preferably substituted with fluoro, preferably difluoro, preferably at the ortho and meta positions relative to the phenyl's attachment to the pyridopyrimidine moiety, or more preferably bromo, preferably at the meta position relative to the phenyl's attachment to the pyridopyrimidine moiety; or more preferably iodo, preferably at the meta position relative to the phenyl's attachment to the pyridopyrimidine moiety.

In another preferred embodiment of compounds containing 8-pyridopyrimidine, the phenyl group is substituted with two or more different halo substituents, preferably disubstituted, and preferably contains fluoro and chloro, and more preferably disubstituted at the non-adjacent ortho and meta positions relative to the phenyl's attachment to the pyridopyrimidine moiety, more preferably where fluoro is at the ortho position and chloro is. at the meta position relative to the phenyl's attachment to the pyridopyrimidine moiety, or preferably is disubstituted with fluoro and bromo, preferably at the non-adjacent ortho and meta positions relative to the phenyl's attachment to the pyridopyrimidine moiety, more preferably where fluoro is at the ortho position and bromo is at the meta position relative to the phenyl's attachment to the pyridopyrimidine moiety.

In another preferred embodiment in compounds containing 8-pyridopyrimidine, the phenyl group is substituted, preferably at one or two positions, and is preferably substituted with alkoxy or arylaryloxy, preferably methoxy, ethoxy isopropoxy, or benzoxy, and preferably at the ortho or meta position relative to the phenyl's attachment to the pyridopyrimidine moiety.

In another embodiment in compounds containing 8-pyridopyrimidine, the phenyl is preferably substituted with alkyl, preferably methyl, and preferably at the meta position relative to the phenyl's attachment to the pyridopyrimidine moiety.

In another preferred embodiment in compounds containing 8-pyridopyrimidine, two or more substituents may join to form a fused ring. Preferably the fused ring is a dioxolane ring, more preferably a 1,3-dioxolane ring, fused to the phenyl ring at the meta and para positions relative to the phenyl's attachment to the pyridopyrimidine moiety.

In another preferred embodiment of compounds containing 8-pyridopyrimidine, the phenyl group is substituted with two or more different substituents, preferably disubstituted, and preferably chloro and methoxy, and preferably disubstituted at the non-adjacent ortho and meta positions relative to the phenyl's attachment to the pyridopyrimidine moiety, more preferably where methoxy is at the ortho position and chloro is at the meta position relative to the phenyl's attachment to the pyridopyrimidine moiety; or preferably is disubstituted with fluoro and methoxy, preferably at the adjacent ortho and meta positions relative to the phenyl's attachment to the pyridopyrimidine moiety, more preferably where fluoro is at the ortho position and methoxy is at the meta position relative to the phenyl's attachment to the pyridopyrimidine moiety.

In addition, in compounds for formula (5) containing the pteridine nucleus, the phenyl group preferably contains at least one halo substituent at the ortho, meta or para positions relative to the phenyl's attachment to the pteridine moiety. In a more preferred embodiment, the phenyl group contains one chloro group at the ortho or meta positions relative to the phenyl's attachment to the pteridine moiety, one fluoro group at the ortho, meta or para positions relative to the phenyl's attachment to the pteridine moiety; or one bromo or iodo at the meta position relative to the phenyl's attachment to the pteridine moiety. In another preferred embodiment, the phenyl group contains two halo groups, preferably difluoro, preferably disubstituted at the non-adjacent ortho and meta positions relative to the phenyl's attachment to the pteridine moiety; preferably dichloro, preferably disubstituted at the adjacent ortho and meta positions relative to the phenyl's attachment to the pteridine moiety; preferably fluoro and chloro, preferably disubstituted at the adjacent or non-adjacent ortho and meta positions relative to the phenyl's attachment to the pteridine moiety, preferably where the fluoro is at the ortho position, and the chloro is at either meta position, and even more preferably where the chloro is at the non-adjacent meta position; or preferably fluoro and bromo preferably substituted at the non-adjacent ortho and meta positions relative to the phenyl's attachment to the pteridine moiety, preferably where the fluoro is at the ortho position, and the bromo is at the non-adjacent meta position.

In another preferred embodiment in compounds containing pteridine, the phenyl group is substituted, preferably at one or more positions, preferably one position, and more preferably with alkoxy, even more preferably with methoxy, and preferably at the ortho or meta position relative to the phenyl's attachment to the pteridine moiety. In another embodiment in compounds containing pteridine, the phenyl is preferably substituted with haloalkyl, preferably trifluoromethyl, and preferably at the meta position relative to the phenyl's attachment to the pteridine moiety.

In another preferred embodiment of compounds of formula (5) containing pteridine, the phenyl group is substituted with two or more different substituents, preferably two substituents, and preferably disubstituted with halo and haloalkyl, more preferably fluoro and trifluoromethyl, and preferably disubstituted at the non-adjacent ortho and meta positions relative to the phenyl's attachment to the pteridine moiety, more preferably where fluoro is at the ortho position and trifluoromethyl is at the meta position relative to the phenyl's attachment to the pteridine moiety.

According to the definition above, R² is a noninterfering substituent. Preferably, R² is independently H, halo, alkyl; alkenyl, alkynyl, acyl 9 or hetero-forms thereof. More preferably R² is lower alkyl (1-3C), halo such as Br, I, Cl or F. Even more preferably, R² is methyl, ethyl, bromo, iodo or CONHR. Most preferably, R² is H.

The following provisos apply to compounds of formula (5):
when Z⁵-Z⁷ are CH and Z⁸ is N, R¹ is not 2-fluoro, 2-chloro or the phenyl is not unsubstituted;
when Z⁵ and Z⁸ are N and Z⁶ and Z⁷ are CH, the phenyl is not unsubstituted; and
when Z⁵ is N and Z⁶-Z⁸ are CH, the phenyl is not unsubstituted.

Representative compound of formula (5) are listed in the following Table 6.

TABLE 6

| Compound # | Structure |
|---|---|
| 297 | 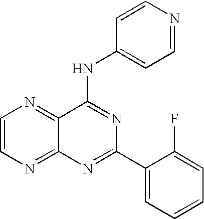 |
| 298 | 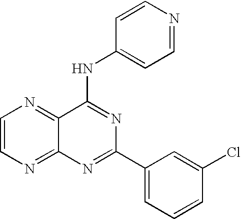 |
| 299 | 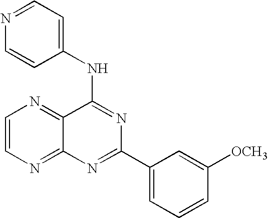 |
| 300 | 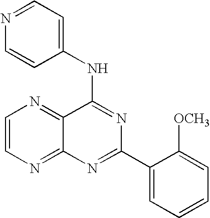 |
| 301 | 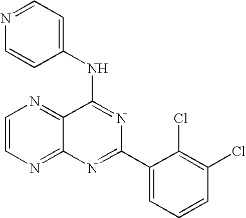 |
| 302 | 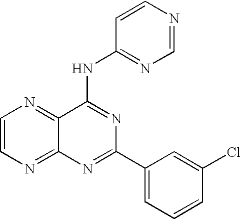 |
| 303 | 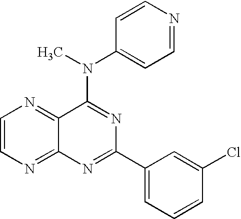 |
| 304 | 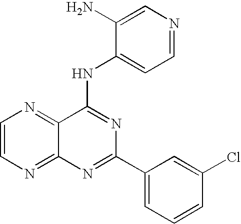 |
| 305 | 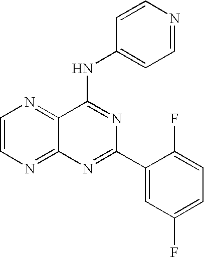 |
| 306 | 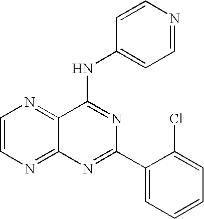 |
| 307 | 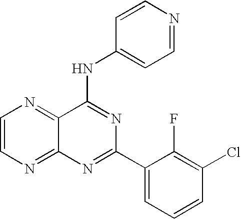 |
| 308 | 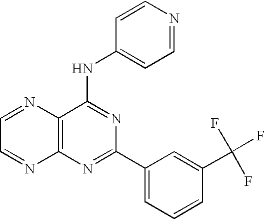 |
| 309 | 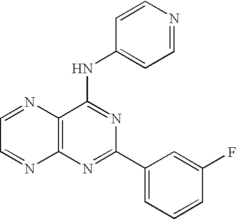 |
| 310 | 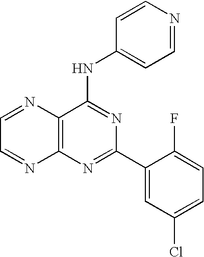 |
| 311 | 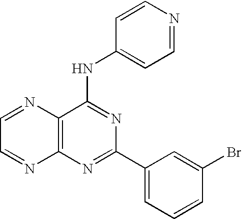 |
| 312 | 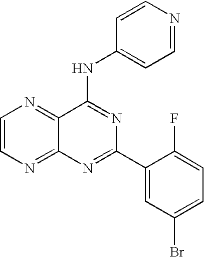 |
| 313 | 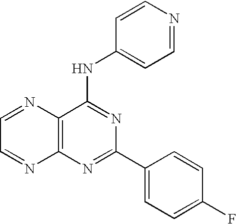 |
| 314 | 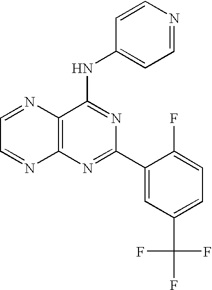 |
| 315 | 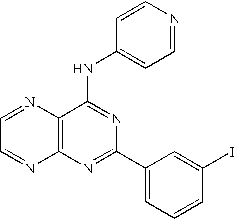 |
| 316 | 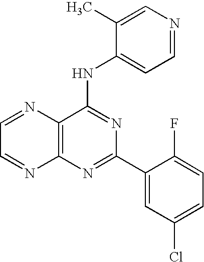 |
| 317 | 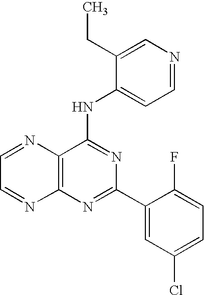 |
| 318 | 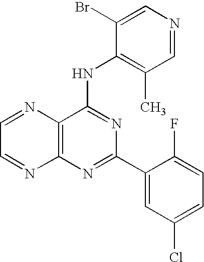 |
| 319 | 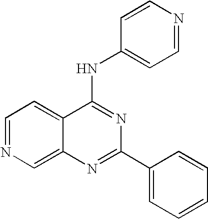 |
| 320 | 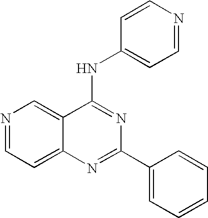 |
| 321 | 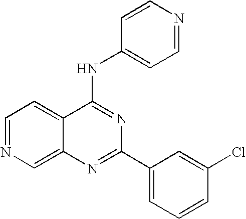 |
| 322 | 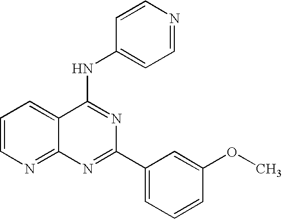 |
| 323 | 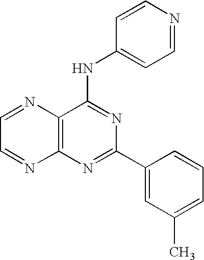 |
| 324 | 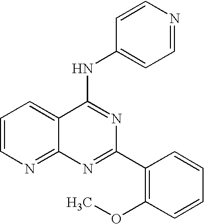 |
| 325 | 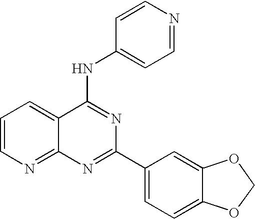 |
| 326 | 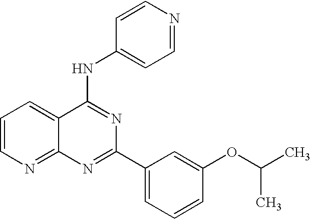 |
| 327 | 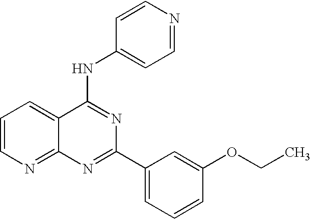 |
| 328 | 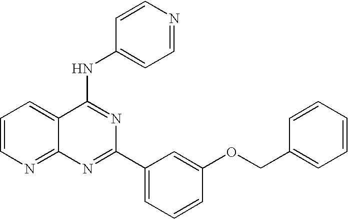 |
| 329 | 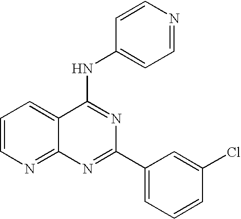 |
| 330 | 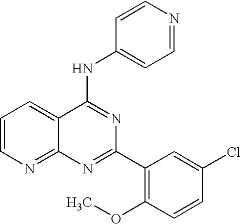 |
| 331 | 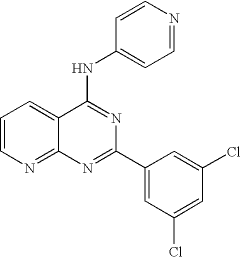 |
| 332 | 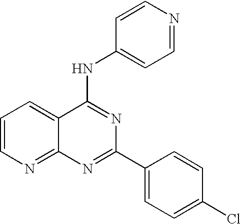 |
| 333 | 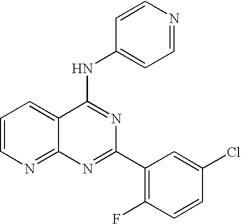 |
| 334 | 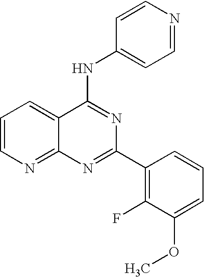 |
| 335 | 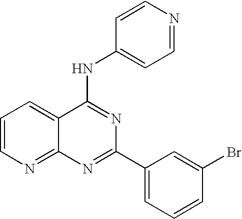 |
| 336 | 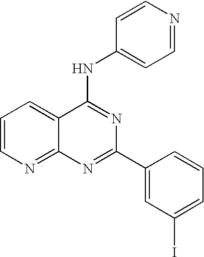 |
| 337 | 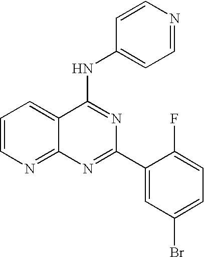 |
| 338 | 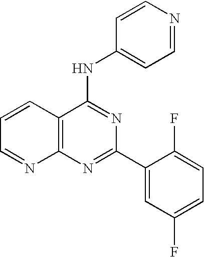 |
| 339 | 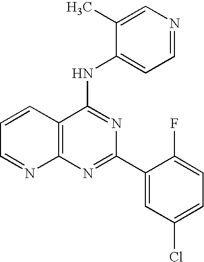 |
| 340 | 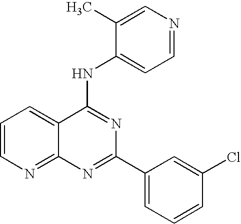 |

The TGF-β inhibitors herein can also be supplied in the form of a “prodrug” which is designed to release the compounds when administered to a subject. Prodrug form designs are well known in the art, and depend on the substituents contained in the compound. For example, a substituent containing sulfnydryl could be coupled to a carrier which renders the compound biologically inactive until removed by endogenous enzymes or, for example, by enzymes targeted to a particular receptor or location in the subject.

In the event that any of the substituents of the foregoing compounds contain chiral centers, as some, indeed, do, the compounds include all stereoisomeric forms thereof, both as isolated stereoisomers and mixtures of these stereoisomeric forms.

The compounds of formulas (1)-(5), may be supplied in the form of their pharmaceutically acceptable acid-addition salts including salts of inorganic acids such as hydrochloric, sulfuric, hydrobromic, or phosphoric acid or salts of organic acids such as acetic, tartaric, succinic, benzoic, salicylic, and the like. If a carboxyl moiety is present on a compound of formula (1)-(5), the compound may also be supplied as a salt with a pharmaceutically acceptable cation.

The compounds of formulas (1)-(5) may also be supplied in the form of a “prodrug” which is designed to release the compounds when administered to a subject. Prodrug formed designs are well known in the art, and depend on the substituents contained in the compounds of formulas (1)-(5). For example, a substituent containing sulfhydryl could be coupled to a carrier which renders the compound biologically inactive until removed by endogenous enzymes or, for example, by enzymes targeted to a particular receptor or location in the subject.

In the event that any of the substituents of the compounds of formulas (1)-(5) contain chiral centers, as some, indeed, do, the compounds include all stereoisomeric forms thereof, both as isolated stereoisomers and mixtures of these stereoisomeric forms.

Synthesis of the Compounds of the Invention

Methods to synthesize the compounds of the invention are, in general, known in the art. Thus, the compounds of the formula (1) may be synthesized as described in WO 00/12497, published on published Mar. 9, 2003. Methods for the synthesis of compounds of formula (2) are disclosed in WO 02/40468 published on May 23, 2002. Compounds of formula (3) can be synthesized, for example, as described in WO 00/61576 published on Oct. 19, 2000. The synthesis of compounds of formula (4) is described, for example, in PCT Application No. PCT/US03/28590. Compounds of formula (5) can be synthesized as described, for example, in U.S. Application No. 60/507,910. In addition, representative compounds within the scope of the invention are further described in U.S. Application No. 60/458,982. The entire disclosures of all documents cited in this section are hereby expressly incorporated by reference.

Activity of the Compounds

Compounds that are useful in the methods of the present invention can be identified by their ability to inhibit TGF-β. An assay for identifying the useful compounds can, for example, be conducted as follows: Compound dilutions and reagents are prepared fresh daily. Compounds are diluted from DMSO stock solutions to 2 times the desired assay concentration, keeping final DMSO concentration in the assay less than or equal to 1%. TGFβ-R1 should be diluted to 4 times the desired assay concentration in buffer+DTT. ATP can be diluted into 4× reaction buffer, and gamma-³³P-ATP can be added at 60 μCi/mL.

The assay can be performed, for example, by adding 10 μl of the enzyme to 20 μl of the compound solution. In a possible protocol, the reaction is initiated by the addition of 10 μl of ATP mix. Final assay conditions include 10 uM ATP, 170 nM TGFβ-R1, and 1M DTT in 20 mM MOPS, pH 7. The reactions are incubated at room temperature for 20 minutes. The reactions are stopped by transferring 23 μl of reaction mixture onto a phosphocellulose 96-well filter plate, which has been pre-wetted with 15 μl of 0.25M H₃PO₄ per well. After 5 minutes, the wells are washed 4× with 75 mM H₃PO₄ and once with 95% ethanol. The plate is dried, scintillation cocktail is added to each well, and the wells are counted in a Packard TopCount microplate scintillation counter.

Alternatively, compounds can be evaluated by measuring their abilities to inhibit the phosphorylation of the substrate casein. An assay can be conducted as follows: Compound dilutions and reagents are prepared fresh daily. Compounds are diluted from DMSO stock solutions to 2 times the desired assay concentration, keeping final DMSO concentration in the assay less than or equal to 1%. TGF-β-R1 kinase should be diluted to 4 times the desired assay concentration in buffer+DTT. ATP and casein can be diluted into 4× reaction buffer, and gamma-33P-ATP can be added at 50 μCi/mL.

According to a possible protocol, the assay can be performed by adding 10 μl of the enzyme to 20 μl of the compound solution. The reaction is initiated by the addition of 10 μl of the casein/ATP mix. Final assay conditions include 2.5 μM ATP, 100 μM casein, 6.4 nM TGF R1 kinase, and 1M DTT in 20 mM Tris buffer, pH 7.5. The reactions are incubated at room temperature for 45 minutes. The reactions are stopped by transferring 23 μl of reaction rmixture onto a phosphocellulose 96-well filter plate, which has been pre-wetted with 15 ul of 0.25M H₃PO₄ per well. After 5 minutes, the wells are washed 4× with 75 mM H₃PO₄ and once with 95% ethanol. The plate is dried, scintillation cocktail is added to each well, and the wells are counted in a Packard TopCount microplate scintillation counter. The ability of a compound to inhibit the enzyme is determined by comparing the counts obtained in the presence of the compound to those of the positive control (in the absence of compound) and the negative control (in the absence of enzyme).

Methods of Treatment

Malignant gliomas that can be treated in accordance with the present invention include, without limitation, astrocytomas, ependymomas, oligodendrogliomas, and mixed gliomas, both in adults and children.

The most common gliomas, astrocytomas start in brain cells called astrocytes and can occur in most parts of the brain (and occasionally in the spinal cord), although they are most commonly found in the cerebrum. Astrocytomas can develop both in adults and children, but are more common in adults. Astrocytomas in the base of the brain are more common in children or young adults. Glioblastoma is a particularly aggressive form of astrocytoma, also referred to as type IV astrocytoma.

Ependymomas are brain tumors that begin in the ependyma, the cells that line the passageways in the brain where the cerebrospinal fluid is made and stored. They are a rare type of glioma and can be found in any part of the brain or spine, but are most commonly found in the cerebrum. Ependymomas may spread from the brain to the spinal cord via the cerebrospinal fluid. People of all ages, including children, can develop ependymomas.

Oligodendrogliomas begin in the brain cells called oligodendrocytes, which provide support and nourishment for the cells that transmit nerve impulses. This type of tumor is normally found in the cerebrum, and can develop both in adults and children.

Mixed gliomas are brain tumors of more than one type of brain cell, including cells of astrocytes, ependymal cells and/or oligodendrocytes. The most common site for a mixed glioma is the cerebrum, but, like other gliomas, they may spread to other parts of the brain. This type of tumor can occur both in adults and children.

Oligodendroglioma is a relatively rare brain-tumor that develops from glial cells called oligodendroglia. There is a malignant form of oligodendroglioma and a mixed malignant astrocytoma-oligodendroglioma, both of which are treated much like the. glioblastoma multiforme.

Optic nerve glioma is found on or near the nerves that travel between the eye and brain vision centers. It is particularly common in people who have neurofibromatosis.

The manner of administration, formulation and dosage of the compounds useful in the invention and their related compounds will depend on the type and severity (grade) of glioma to be treated, the particular subject to be treated, and the judgement of the practitioner; formulation will depend on mode of administration.

Current treatment of glioblastomas include surgery followed by radiation and/or chemotherapy.

The compounds of the invention are conveniently administered by oral administration by compounding them with suitable pharmaceutical excipients so as to provide tablets, capsules, syrups, and the like. Suitable formulations for oral administration may also include minor components such as buffers, flavoring agents and the like. Typically, the amount of active ingredient in the formulations will be in the range of about 5%-95% of the total formulation, but wide variation is permitted depending on the carrier. Suitable carriers include sucrose, pectin, magnesium stearate, lactose, peanut oil, olive oil, water, and the like.

The compounds may also be administered by injection, including intravenous, intramuscular, subcutaneous, intrarticular, intraperitoneal, or intracranial injection. Typical formulations for such use are liquid formulations in isotonic vehicles such as Hank's solution or Ringer's solution.

In general, any suitable formulation may be used. A compendium of art-known formulations is found in Remington's Pharmaceutical Sciences, latest edition, Mack Publishing Company, Easton, Pa. Reference to this manual is routine in the art.

The dosages of the compounds of the invention will depend on a number of factors which will vary from patient to patient. However, it is believed that generally, the daily oral dosage will utilize 0.001-100 mg/kg total body weight, preferably from 0.01-50 mg/kg and more preferably about 0.01 mg/kg-10 mg/kg body weight. The dose regimen will vary, however, depending on the particular tumor to be treated, the age, sex, and overall condition of the patient, and the judgment of the practitioner.

It should be noted that the compounds useful for the invention can be administered as individual active ingredients, or as mixtures of several different compounds. In addition, the TGF-β inhibitors can be used as single therapeutic agents or in combination with other therapeutic agents. Drugs that could be usefully combined with these compounds include natural or synthetic corticosteroids, particularly prednisone and its derivatives, monoclonal antibodies targeting cells of the immune system or genes associated with the development or progression of malignant gliomas, and small molecule inhibitors of cell division, protein synthesis, or mRNA transcription or translation, or inhibitors of immune cell differentiation or activation.

In particular, the compounds of the present invention can be administered as part of a treatment regimen that may include radiotherapy, administration of other chemotherapeutic agents, immunotherapy or steroid therapy, bone marrow transplantation, and other treatment options, in any combination and order determined by the physician.

As implicated above, although the compounds of the invention may be used in humans, they are also available for veterinary use in treating non-human mammalian subjects.

Further details of the invention will be apparent from the following non-limiting examples.

### EXAMPLE

Inhibition the Growth of Glioma In Vitro and In Vivo with a TGF-β Inhibitor

The effects of TGFβ-R1 kinase inhibitors, specifically, of the inhibitor designated as Compound No. 79 in Table II, on the growth and immunogenicity of murine SMA-560 and human LN-308 glioma cells and the growth of, and immune response to, intracranial SMA-560 gliomas in syngeneic VM/Dk mice in vivo was studied.

Materials and Methods

Cell Lines and Reagents

Compound No. 79 is a TGF-βRI kinase inhibitor developed by Scios Inc. Phytohemagglutinin (PHA) was from Biochrom (Berlin, Germany). [Methyl-³H]-thymidine as obtained from Amersham (Braunschweig, Germany). ⁵¹Cr was purchased from New England Nuclear (Boston, Mass.). Human recombinant TGF-β₁ and TGF-β₂ were obtained from Peprotech (London, UK). Mouse IL-2 was from Peprotech (London, UK). Neutralizing pan-anti-TGF-β antibody was purchased from R & D (Wiesbaden, Germany). The human malignant glioma cell line LN-308 was kindly provided by N. de Tribolet (Centre Hospitalier Universitaire Vaudois, Lausanne, Switzerland). The murine glioma line SMA-560 was a kind gift of D. D. Bigner (Duke University Medical Center, Durham, N.C.). CCL64 mink lung epithelial cells were obtained from the American Type Culture Collection (Rockville, Md.).

Cell Culture

The glioma cells and CCL64 cells were maintained in DMEM supplemented with 2 mM L-glutamine (Gibco Life Technologies, Paisley, UK), 10% FCS (Biochrom KG, Berlin, Germany) and penicillin (100 IU/ml)/streptomycin (100 μg/ml) (Gibco). Growth and viability of the glioma cells was examined by crystal violet, LDH release (Roche, Mannheim, Germany) and trypan blue dye exclusion assays. To assess clonogenicity, 500 SMA-560 cells were seeded into 6 well plates (9.4 cm²). After formation of visible cell formations, colonies>20 cells were counted.

Human PBMC were isolated from healthy donors by density gradient centrifugation (Biocoll, Biochrom KG). Monocytes were depleted by adhesion and differential centrifugation to obtain peripheral blood lymphocytes (PBL). To obtain purified T cells, the PBMC were depleted of B cells and monocytes using LymphoKwik T™ reagent (One Lambda Inc., Canoga Park, Calif.). The purity of this population was verified by flow cytometry using anti-human CD3-PE antibody to be>97% (Becton Dickinson, Heidelberg, Germany). Human polyclonal NK cell populations were obtained by culturing PBL on irradiated RPMI8866 feeder cells for 10 days (Valiante et al., Cel.. Immunol., 145:187-198 (1992)). Murine NK cells were prepared from splenocytes from VM/Dk mice by positive selection using DX5 monoclonal antibody-coupled magnetic beads with the corresponding column system (Miltenyi Biotech, Bergisch Gladbach, Germany) and cultured with mouse IL-2 (5000 U/ml) for at least 10 days before use. The human polyclonal NK cell cultures, human PBL, human T cells and mouse NK cells were grown in RPMI 1640 supplemented with 15% FCS, 2 mM L-glutamine, 1 mM sodium pyruvate, 50 μM β-mercaptoethanol and penicillin (100 IU/ml)/streptomycin (100 μg/ml).

TGF-β Bioassay

The levels of bioactive TGF-β were determined using the CCL64 bioassay. Briefly, 104 CCL64 cells were adhered to 96 well plates for 24 h, full medium was replaced by serum-free medium, and the cells were exposed to recombinant TGF-β1/2 or glioma cell culture supernatants diluted in serum-free medium for 72 h. Growth was assessed by crystal violet staining at 72 h. Glioma cell supernatants were harvested from confluent cultures maintained for 48 h in serum-free medium and heat-treated (5 min, 85° C.) to activate latent TGF-β (Leitlein et al., J. Immunol., 166:7238-7243 (2001)).

Immunoblot Analysis

The levels of phosphorylated Smad2 (p-Smad2) protein levels were analyzed by immunoblot using 20 μg of protein per lane on a 12% acrylamide gel. After transfer to a PVDF membrane (Amersham, Braunschweig, Germany), the blots were blocked in PBS containing 5% skim milk and 0.05% Tween 20, and incubated overnight at 4° C. with p-Smad2 antibody (2 μg/ml). Visualization of protein bands was accomplished using horseradish peroxidase-coupled secondary antibody (Sigma) and enhanced chemiluminescence (Amersham). Total Smad2/3 levels were assessed using a specific Smad2/3 antibody (Becton-Dickinson).

Lysis Assay

HLA-A2-mismatched PBL or T cells (107/25 cm2 flask) were cocultured with 106 irradiated (30 Gy) glioma cells for 5 days. Glioma cell targets were labeled using 51Cr (50 μCi, 90 min) and incubated (104/well) with effector PBL harvested from the cocultures at effector:target (E:T) ratios of 100:1 to 3:1. The maximum 51Cr release was determined by addition of NP40 (1%). After 4 h the supernatants were transferred to a Luma-PlateTM-96 (Packard, Dreieich, Germany) and measured. The percentage of 51Cr release was calculated as follows: 100×([experimental release−spontaneous release]/[maximum release−spontaneous release]).

Cytokine Release

IL-10, TNF-α and IFN-γ release by immune effector cells was assessed by Elispot assay in a multiscreen-HA 96 well plate (Millipore, Eschborn, Germany), coated with corresponding anti-human capture antibodies (Becton Dickinson). Briefly, 5×104 glioma cells were cocultured for 24 h with 105, 2.5×105 or 5×105 HLA-A2-mismatched, prestimulated (5 days) PBL. The cells were removed using double-distilled water, and captured cytokines were visualized using biotinylated antibodies and streptavidin-alkaline phosphatase (Becton Dickinson). Spots were counted on an Elispot reader system (AID, Straβberg, Germany). Similarly, freshly isolated splenocytes were assayed for IFN-γ release in ex vivo experiments, using anti-mouse capture IFN-γ antibody and the corresponding biotinylated secondary antibody (Becton Dickinson).

Flow Cytometry

The adherent glioma cells were detached nonenzymatically using cell dissociation solution (Sigma). Cell cycle analysis was performed by using fixed and permeabilized glioma cells (70% ethanol). RNA was digested with RNase A (Life Technologies, Inc.). DNA was stained with propidium iodide (50 μg/ml).

Animal Experiments

VM/Dk mice were purchased from the TSE Research Center (Berkshire, UK). Mice of 6-12 weeks of age were used in all experiments. The experiments were performed according to the German animal protection law. Groups of 7-8 mice were anesthesized before all intracranial procedures and placed in a stereotaxic fixation device (Stoelting, Wood Dale, Ill.). A burr hole was drilled in the skull 2 mm lateral to the bregma. The needle of a Hamilton syringe (Hamilton, Darmstadt, Germany) was introduced to a depth of 3 mm. Five×103 SMA-560 cells (Serano et al., Acta Neuropathol., 51:53-64 (1980)) resuspended in a volume of 2 μl PBS were injected into the right striatum. Three days later the mice were allowed to drink Compound No. 79 dissolved at 1 mg/ml in deionized water. The mice were observed daily and, in the survival experiments, sacrified when developing neurological symptoms, or sacrificed as indicated in the other experiments.

Ex Vivo Immune Effector Assays

Glioma-bearing mice were sacrificed 10 days after tumor cell injection. Splenocytes were isolated and used in 24 h IFN-γ Elispot assays as described above. Further, those cells were stimulated with IL-2 (5000 U/ml) for 10 days to generate LAK cells which were used in 51Cr release assays against SMA-560 glioma cells as targets.

Statistical Analysis

The experiments were usually performed at least three times with similar results. Significance was tested by Student's t-test. P values are derived from two-tailed t-tests.

Results

Compound No. 79 is a TGF-β1 and TGF-β2 Inhibitor In Vitro

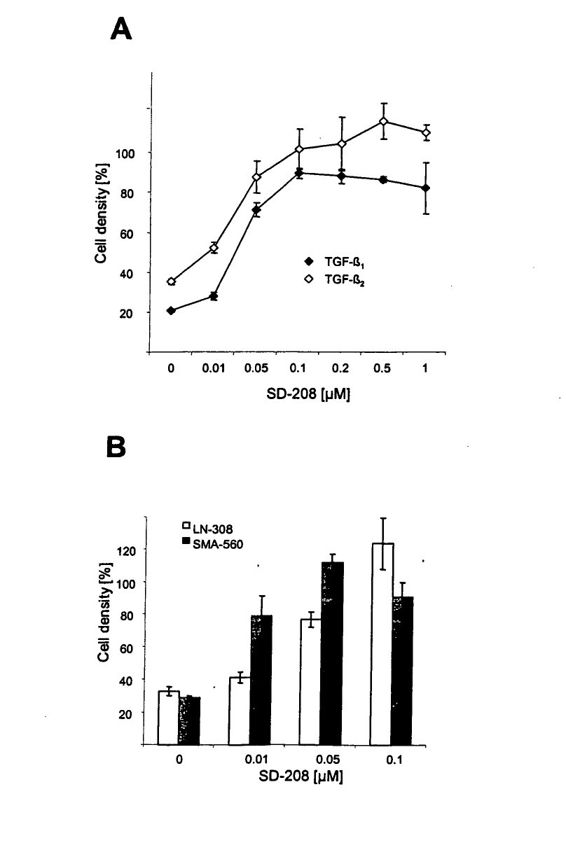

CCL64 mink lung epithelial cells are sensitive to the growth inhibitory effects of human TGF-β1 and TGF-β2 at EC50 concentrations of 0.5 ng/ml. The inhibitory effects of recombinant TGF-β as well as those of TGF-β-containing glioma cell supernatants are abrogated by specific TGF-β antibodies (Leitlein et al., J. Immunol. 166:7238-7243 (2001), and data not shown). The CCL64 bioassay was used here to verify the TGF-β-antagonistic properties of Compound No. 79. Compound No. 79 rescued the inhibition of growth mediated by TGF-β1 or TGF-β2 (10 ng/ml) in a concentration-dependent manner, with an EC50 concentration in the range of 0.03 μM (FIG. 1A). Similarly, the growth inhibition mediated by diluted serum-free SMA-560 or LN-308 glioma cell supematants was nullified by the same concentrations of Compound No. 79 (FIG. 1B).

Compound No. 79 Abrogates TGF-β-dependent Signal Transduction in Glioma Cells

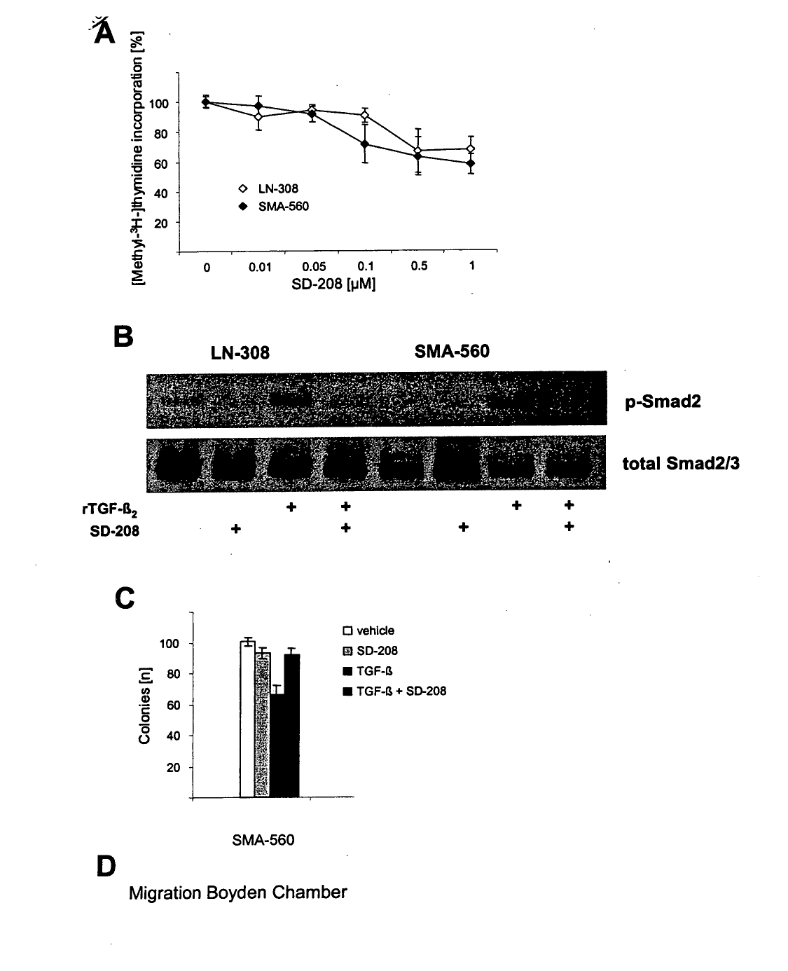

Next, the biological effects of Compound No. 79 on murine and human glioma cells were examined in vitro. The concentrations required to block the growth inhibitory effects of TGF-β in the CCL64 bioassay had no effect on the proliferation of either cell line. However, higher concentrations of up to 1 μM moderately inhibited the growth of both cell lines (FIG. 2A). Inhibition of growth was related to impaired proliferation, but not actual cell death, since neiter LDH release nor trypan blue dye exclusion assays revealed cytotoxic effects of Compound No. 79 on the glioma cells at concentrations up to 1 μM for 72 h. Flow cytometric cell cycle analysis performed at 48 h after exposure to Compound No. 79 at 0.01, 0.1 or 1 μM revealed no specific type of cell cycle arrest in either cell line (data not shown). Moreover, Compound No. 79 did not modulate the viability of the glioma cells in response to serum deprivation (data not shown). However, the inhibition of signaling transduced by endogenous or exogenous TGF-β was ascertained by demonstrating that Compound No. 79 interfered with Smad2 phosphorylation without altering total cellular Smad2/3 levels (FIG. 2B). Of note, pSmad phosphorylation in untreated glioma cells was barely detectable, but this signal was also abolished by compound 79.

Compound No. 79 Enhances Allogeneic Immune Responses to Glioma Cells In Vitro

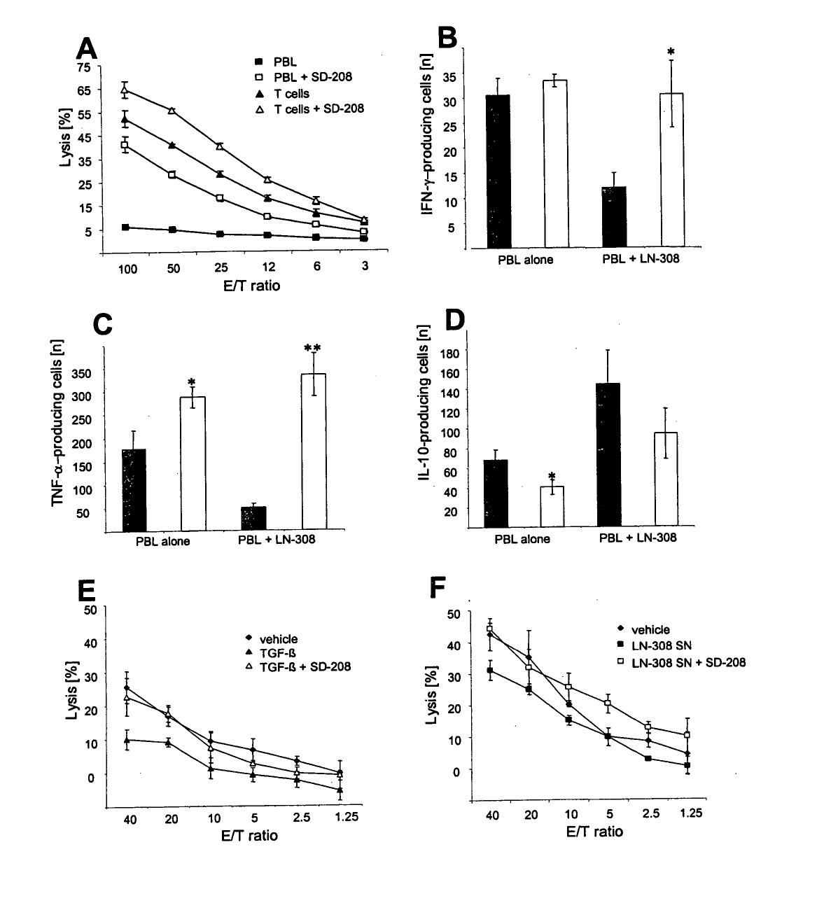

The next series of experiments was designed to examine whether Compound No. 79 restores allogeneic immune cell responses to cultured human glioma cells. When HLA-A2-mismatched PBL or purified T cells were cocultured with irradiated glioma cells in the absence or presence of Compound No. 79, their lytic activity in a subsequent 4 h ⁵¹Cr release assay was significantly enhanced by a preexposure to Compound No. 79 (FIG. 3A). Similar effects were obtained using neutralizing TGF-β antibodies (10 μg/ml, added every two days) (data not shown). The release of IFN-γ by HLA-mismatched PBL was strongly inhibited when the priming had taken place in the presence of glioma cells. Compound No. 79 restored the IFN-γ release to levels comparable to PBL pre-cultured in the absence of LN-308 cells (FIG. 3B). Similar results were obtained for TNF-α (FIG. 3C). In contrast, IL-10 release was stimulated after coculturing with LN-308 cells, and Compound No. 79 reduced the release of IL-10 by immune effector cells generated both from unstimulated and glioma cell-primed cultures (FIG. 3D).

The lytic activity against LN-308 targets of polyclonal NK cells was strongly inhibited by exogenous TGF-β, and TGF-β-mediated inhibition was relieved by Compound No. 79 (FIG. 3E). Similarly, LN-308 cell supernatants inhibited NK cell activity, and this inhibition was also blocked by Compound No. 79 (FIG. 3F) or neutralizing TGF-β antibodies (data not shown).

Compound No. 79 Prolongs the Survival of SMA-560 Intracranial Experimental Glioma-bearing Syngeneic Mice

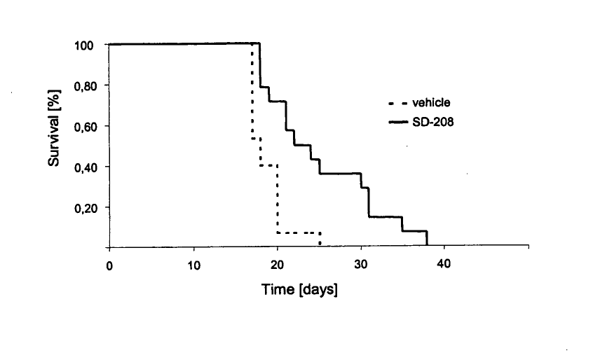

The therapeutic effects of Compound No. 79 administered via the drinking water (1 mg/ml) were assessed in the syngeneic SMA-560 mouse glioma paradigm (Friese et al. 2003). The development of neurological symptoms was delayed in Compound No. 79-treated mice (data not shown) and mean survival was prolonged to 25.1±6.5 days (median 23) compared with 18.6±2.1 days (median 18) in vehicle-treated animals (FIG. 4) (p=0.004, t-test). The survival rate at 30 days was 29% in Compound No. 79-treated animals, but 0% in control animals.

Compound No. 79 Modulates Immune Responses to SMA-560 Glioma Cells In VivO

Elispot assays for IFN-γ release by splenocytes harvested at day 7 after the initiation of treatment with Compound No. 79 revealed an increase over background in 3 of 5 Compound No. 79-treated animals, but only 1 of 5 control animals (FIG. 4B). Further, LAK cells generated from the splenocytes of Compound No. 79-treated animals showed an enhanced lytic activity against SMA-560 as targets (FIG. 4C).

Discussion

Antagonizing the biological effects of TGF-β has become one of the major strategies to combat various types of cancer including malignant gliomas. Current rationales for anti-TGF-β strategies include its putative role in migration and invasion (Wick et al., J. Neurosci. 21:3360-3368 (2001)), metastasis (Yang et al., J. Clin. Invest., 109:1607-1615 (2002)) and tumor-associated immunosuppression (Weller and Fontana, Brain Res. Rev., 21:128-151 (1995); Gorelik and Flavelli, Nat. Rev. Immunol., 2:46-53 (2002)). All of the TGF-β-based therapeutic approaches evaluated in experimental gliomas so far appear to have limitations with regard to their transfer into the clinic. Antisense oligonucleotides pose severe problems in terms of delivery to the desired site of action. The same applies to gene therapy strategies based e.g. on the transfer of the decorin gene (Ständer et al., Gene Ther., 5:1187-1194 (1998)). Inhibition of furin-like proteases aiming at limiting TGF-β bioactivity at the level of TGF-β processing (Leitlein et al., J. Immunol. 166:7238-7243 (2001)) may not be achieved with acceptable specificity at present since a whole variety of molecules require processing by such enzymes (Thomas, G., Nat. Rev. Mol. Cell Biol. 3:753-766 (2002)). More specificity may result from the use of soluble TGF-β receptor fragments which act to scavenge bioactive TGF-β before it may reach the target cell population [Yang et al., supra; Muraoka et al., J. Clin. Invest. 109:1551-1559 (2002)). This effect should in theory be mimicked by specific small molecules designed to protect cells from the actions of TGF-β at the level of intracellular signal transduction.

Here the activity of one such candidate agent, Compound No. 79, is characterized against murine and human glioma cells in vitro and in vivo. Human LN-308 cells were chosen because they are paradigmatic for their prominent TGF-β synthesis (Fontana et al. supra; Leitlein et al., supra). SMA-560 cells transplanted in syngeneic VM/Dk mice represent the best model for the immunotherapy of rodent gliomas (Serano et al., Acta Neuropathol., 51:53-64 (1980)). The experiments confirmed that Compound No. 79 is a potent antagonist of TGF-β₁ and TGF-β₂ in the CCL64 mink lung epithelial assay (FIG. 1A) and abrogates the inhibitory effects of glioma cell SN on the growth of these cells (FIG. 1B). Compound No. 79 is not cytotoxic to glioma cells and only moderately inhibits proliferation at higher concentrations (FIG. 2A). In that regard, a negative growth regulatory effect of TGF-β on SMA-560 cells has not been confirmed (Ashley et al., Cancer Res., 15:302-309 (1998)). Smad2 phosphorylation is rapidly induced by TGF-β in a manner sensitive to Compound 79 (FIG. 2B), indicating that TGF-β signaling is not abrogated constitutively in glioma cells, but may not play a role in the modulation of glioma cell proliferation. Moreover, the antagonism of autocrine and paracrine signaling by TGF-β in glioma cells treated with Compound No. 79 predicts that Compound No. 79-like agents may also be potent inhibitors of migration and invasion in glioma cells (Wick et al., J. Neurosci., 21:3360-3368 (2001)).

The work then focused on the desired immune modulatory effect of Compound No. 79 which should result in an enhanced immunogenicity of glioma cells as a consequence of reduced TGF-β bioactivity. Human PBL and purified T cells developed enhanced lytic activity against LN-308 glioma cell targets when prestimulated with glioma cells in the presence of Compound No. 79 (FIG. 3A). This was paralleled by an enhanced release of proinflammatory cytokines such as IFN-γ and TNF-α and a reduced release of the immunosuppressive cytokine IL-10 in Compound No. 79-treated cells (FIG. 3B-D). Similarly Compound No. 79 restored the lytic activity of polyclonal NK cell cultures cocultured with TGF-β₁ or LN-308 SN (FIG. 3E-F).

Compound No. 79 prolonged the median survival of SMA-560 glioma-bearing mice significantly (FIG. 4A). No dose-limiting toxicity has been reached, but higher doses could not be administered by the drinking water because of relatively poor solubility of Compound No. 79, suggesting that the therapeutic effect of Compound No. 79 or related agents might even be improved in that glioma model. Without being limited to any particular theory or mechanism, the therapeutic effect of Compound No. 79 might be mediated by the inhibition of glioma cell migration and invasion (Wick et al., supra) or the promotion of anti-glioma immune responses (Weller and Fontana, supra). In support of the latter, ex vivo analyses of splenocytes from Compound 79-treated, glioma-bearing mice revealed enhanced IFN-γ release as well as an increase in LAK activity not lost after 10 days in culture (FIG. 4B,C).

The present data strongly suggest a role for Compound No. 79 or related molecules in the treatment of gliomas. Such a systemic treatment with TGF-βRI antagonists might well be combined with local approaches to limit the bioavailability of TGF-β, e.g., TGF-β antisense oligonucleotides.

All references cited throughout the specification are expressly incorporated herein by reference. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, and the like. All such modifications are within the scope of the claims appended hereto.

1. A method for the treatment of a malignant glioma in a mammalian subject comprising administering to said subject a therapeutically effective amount of a molecule that specifically binds to a TGFβ-R1 kinase receptor.

2. The method of claim 1 wherein said glioma is selected from the group consisting of astrocytomas, ependymomas, oligodendrogliomas, mixed gliomas, oligodendrogliomas, and optic nerve gliomas.

3. The method of claim 2 wherein said glioma is an astrocytoma.

4. The method of claim 3 wherein said astrocytoma is glial myoblastoma.

5. The method of claim 1 wherein said mammal is a human.

6. The method of claim 5 wherein said human is an adult.

7. The method of claim 6 wherein said human is a child.

8. The method of claim 1 wherein said molecule is a non-peptide small molecule.

9. The method of claim 1 wherein said molecule additionally inhibits a biological activity mediated by a p38 kinase.

10. The method of claim 1 wherein said molecule preferentially inhibits a biological activity mediated by TGF-β-R1 kinase relative to a biological activity mediated by p38 kinase.

11. The method of claim 1 wherein said molecule is a compound of formula (1) 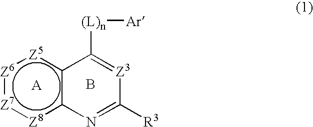
and the pharmaceutically acceptable salts and prodrug forms thereof
wherein R³ is a noninterfering substituent;
each Z is CR² or N, wherein no more than two Z positions in ring A are N, and
wherein two adjacent Z positions in ring A cannot be N;
each R² is independently a noninterfering substituent;
L is a linker;
n is 0 or 1; and
Ar′ is the residue of a cyclic aliphatic, cyclic heteroaliphatic, aromatic or heteroaromatic moiety optionally substituted with 1-3 noninterfering substituents, or a pharmaceutically acceptable salt or prodrug form thereof.

12. The method of claim 11 wherein said compound is a quinazoline derivative.

13. The method of claim 11 wherein Z³ is N; and Z⁵-Z⁸ are CR².

14. The method of claim 11 wherein Z³ is N; and at least one of Z⁵-Z⁸ is nitrogen.

15. The method of claim 11 wherein R³ is an optionally substituted phenyl moiety.

16. The method of claim 11 wherein R³ is selected from the group consisting of 2-, 4-, 5-, 2,4- and 2,5-substituted phenyl moieties.

17. The method of claim 11 wherein R³ is substituted by at least one alkyl(1-6C), alkoxy(1-6C) or halo.

18. The method of claim 11 wherein said compound of formula (1) is [4-(3-methyl)-pyridyl]-6-chloro-2-fluorophenyl-pyridine, or a pharmaceutically acceptable salt of prodrug form thereof.

19. The method of claim 1 wherein said molecule is a compound of formula (4) 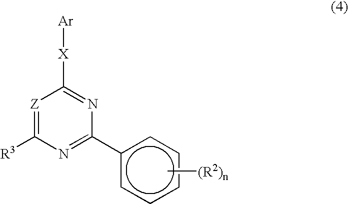
wherein:
Ar represents an optionally substituted aromatic or optionally substituted heteroaromatic moiety containing 5-12 ring members wherein said heteroaromatic moiety contains one or more O, S, and/or N with a proviso that the optionally substituted Ar is not 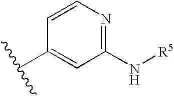
wherein R5 is H, alkyl (1-6C), alkenyl (2-6C), alkynyl (2-6C), an aromatic or heteroaromatic moiety containing 5-11 ring members;
X is NR¹, O, or S;
R¹ is H, alkyl (1-8C), alkenyl (2-8C), or alkynyl (2-8C);
Z represents N or CR⁴;
each of R³ and R⁴ is independently H, or a non-interfering substituent; each R² is independently a non-interfering substituent; and
n is 0, 1, 2, 3, 4, or 5;
or a pharmaceutically acceptable salt or a prodrug form thereof.

20. The method of claim 19, wherein if n>2, and the R²'s are adjacent, they can be joined together to form a 5 to 7 membered non-aromatic, heteroaromatic, or aromatic ring containing 1 to 3 heteroatoms where each heteroatom can independently be O, N, or S.

21. The method of claim 1 wherein said molecule is a compound of formula (5): 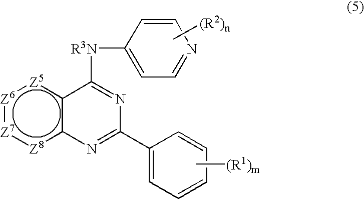
wherein:
each of Z⁵, Z⁶, Z⁷ and Z⁸ is N or CH and wherein one or two Z⁵, Z⁶, Z⁷ and Z⁸ are N and wherein two adjacent Z positions cannot be N;
m and n are each independently 0-3;
R¹ is halo, alkyl, alkoxy or alkyl halide and wherein two adjacent R¹ groups may be joined to form a heterocyclic ring of 5-6 members;
R² is a noninterfering substituent; and
R³ is H or CH₃,
or a pharmaceutically acceptable salt or a prodrug form thereof.

22. A method for reversing a TGF-β-mediated effect on a gene associated with a malignant glioma, comprising contacting a cell comprising said gene with a non-peptide small molecule inhibitor of TGF-β that specifically binds to a TGFβ-R1 receptor kinase present in said cell.

23. The method of claim 22 wherein said cell is associated with glioblastoma.

24. The method of claim 22 wherein said gene is over-expressed in said cell.

25. The method of claim 22 wherein said gene is under-expressed in said cell.

26. The method of claim 22 wherein said inhibitor reverses the TGF-β-mediated effect on the expression of two or more genes.

27. The method of claim 22 wherein said inhibitor reverses the TGF-β-mediated effect on the expression of a multiplicity of genes associated with glioblastoma.

28. The method of claim 22 wherein said gene or genes is/are selected from the group consisting of TGF-β₁, TGF-β₂, TGF-β₃, TGF-β RI, TGF-β RII, Smad2, Smad3, Smad4, IL-10, CD95, IL-6, Il-1, IGF-1, VEGF, MMP, COX-2, TIPM, PAI-1, TNFα, IL-11, EG, and FGF.

29. The method of claim 22 wherein said inhibitor additionally blocks biological activities mediated by Smad proteins, p38 and TAK1.
